(19) United States
(12) Patent Application Publication
(10) Pub. No.: US 2010/0041617 A1
(43) Pub. Date: Feb. 18, 2010

(54) Modulating mxa expression

(76) Inventors: Jane Trepel, Bethesda, MD (US); Alexandra Lin, Oak Hills, VA (US); Sunmin Lee, Boyds, MD (US); Chand Khanna, Chevy Case, MD (US); Min-Jung Lee, Rockville, MD (US); Eun J. Chung, Rockville, MD (US); Ruilli Huang, Rockville, MD (US); David G. Covell, Chevy Chase, MD (US)

Correspondence Address: SCHWEGMAN, LUNDBERG & WOESSNER, P.A., P.O. BOX 2938, MINNEAPOLIS, MN, 55402, US

(21) Appl. No.: 11/663,936

(22) PCT Filed: Sep. 27, 2005
(86) PCT No.: PCT/US05/34849

§ 371 Date: Sep. 15, 2009

Related U.S. Application Data
(60) Provisional application No. 60/613,371, filed on Sep. 27, 2004.

(30) Foreign Application Priority Data
Oct. 1, 2004 (IT) M12004A001869

(51) Int. Cl. A61K 31/7048; A61K 31/428; A61K 31/473; A61K 31/437; A61K 31/397; C12Q 1/68; C07K 14/00; A61P 35/00

(52) U.S. Cl. 514/27; 514/367; 514/297; 514/287; 514/210.02; 435/6; 530/350

(57) ABSTRACT

The invention provides compositions and methods for inhibiting cell motility, metastatic cancer and viral infections in a mammal that involve increasing the activity or expression of MxA.

This application claims benefit of the filing date of U.S. Provisional Ser. No. 60/613,371, filed Sep. 27, 2004, the contents of which are incorporated herein by reference.

### GOVERNMENT RIGHTS

The invention described herein was developed with support from the National Institutes of Health. The U.S. Government has certain rights in the invention.

### FIELD OF THE INVENTION

The invention relates to agents that can modulate the expression of MxA, a gene whose expression can modulate cellular motility and invasiveness of mammalian tumor cells.

### BACKGROUND OF THE INVENTION

Malignant cancer tumors shed cells that migrate to new tissues and create secondary tumors. A benign tumor does not generate secondary tumors. The process of generating secondary tumors is called metastasis and is a complex process in which tumor cells colonize sites distant from the primary tumor. Tumor metastasis remains the major cause of morbidity and death for patients with cancer. One of the greatest challenges in cancer research is to understand the basis of metastasis, i.e., what controls the spread of tumor cells through the blood and lymphatic systems and what allows tumor cells to populate and flourish in new locations.

While surgery and chemotherapy are routinely used for treating cancer, such treatments typically involve removal or ablation of significant tissue, often giving rise to undesirable side effects. Moreover, the surgeon is rarely certain that all malignant tissues are removed. Hence, new compositions and methods for reducing the metastasis of cancer cells are needed.

### SUMMARY OF THE INVENTION

The invention is directed to compositions and methods for modulating MxA expression to control cell migration, including cancer cell metastasis, and viral infection.

One aspect of the invention is a method of inhibiting cell migration in a mammal by administering to the mammal an effective amount of an agent that can increase the expression or activity of MxA in the mammal. The invention is also directed to compositions of these agents, where the composition contains a pharmaceutically acceptable carrier and a therapeutically effective amount of the agent. The composition can be formulated for administration by oral or parenteral routes. In some embodiments, the composition is formulated for local administration to the site of a tumor. In further embodiments, the composition is formulated for sustained release after local or systemic administration.

Another aspect of the invention is a method of treating or preventing metastatic cancer in a mammal comprising administering to the mammal an effective amount of an agent that increases the expression or activity of MxA in the mammal.

In some embodiments, the agent that can increase the expression of MxA is a compound of formula I or a pharmaceutically acceptable salt thereof.

R₁—X(R₃)—R₂ I

wherein:

X is methylene (CH₂), nitrogen or oxygen;
R₁ and R₂ are cycloalkyl, aryl, arylalkylene, heteroaryl, heterocyclyl, or alkyl, any of which may be substituted with oxygen (O), hydroxyl (OH), sulfite (SO₃), sulfate (SO₄), sulfonamide (NH—SO₂ or NH—SO₃), halogen (F, Cl, Br, or I), carboxylate (CO₂), nitro (NO₂), amino (NH₂), secondary or tertiary alkylamino, alkylsulfonamide, lower alkyl, cycloalkyl, alkylenehydroxy, alkoxy, alkoxycarbonyl, alkoxyalkylenecarboxylic acid, alkylenecarboxylic acid, alkyleneaminoalkylene, alkyleneaminoalkylenehydroxy, alkanoyloxy, aminoaryl or aryl; and

R₃ is nothing, hydrogen or, together with an X nitrogen to which it is attached, forms a heterocyclic ring with 0-2 double bonds between the carbon atoms of the heterocyclic ring or 0-1 additional nitrogen atoms.

For example, in some embodiments, R₁ and R₂ are separately selected from the following benzothiozolyl, acridinyl, indolyl, xanthenyl, nitrophenyl or triethylaminyl.

The following compounds are examples of compounds of formula I:

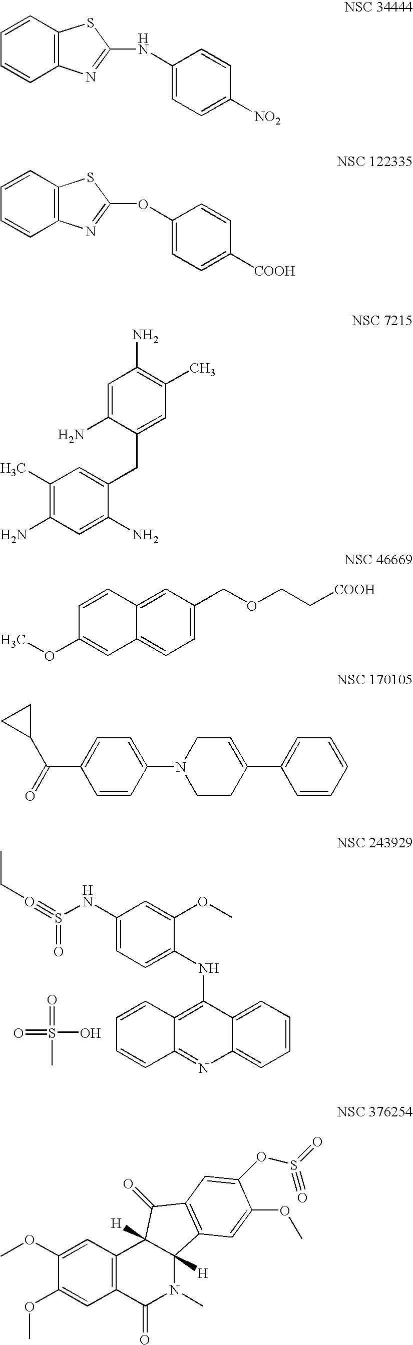

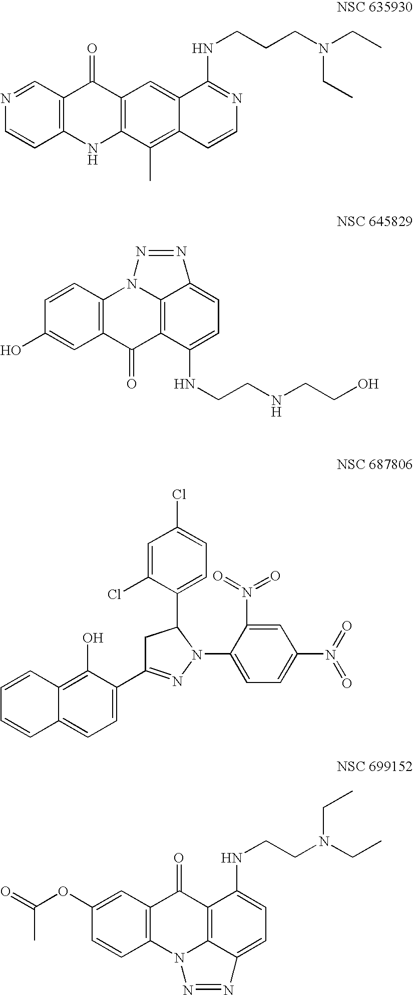

In another embodiment, the compounds of the invention are benzopyrene-saccharide compounds (II). The following are examples of the benzopyrene saccharide compounds of the invention:

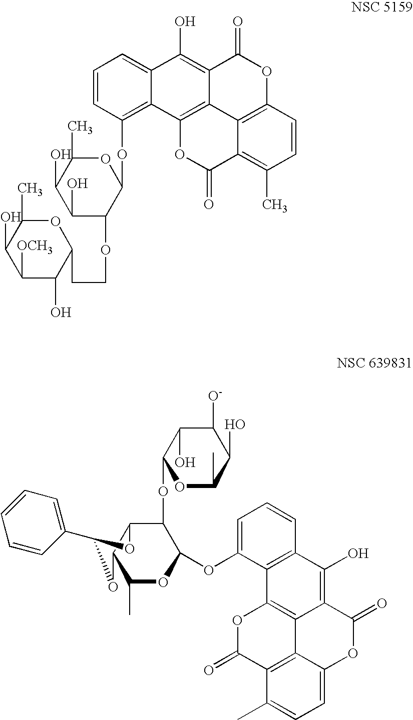

Other agents that can increase the expression of MxA in mammals or mammalian cells include compounds such as: NSC690269, NSC692406, NSC692407, NSC697537, NSC699152, NSC699167, NSC699491, NSC699782, NSC699881, NSC701744, NSC704660, NSC706453, NSC708444, NSC713080, NSC715435, NSC716204, NSC717200, NSC718885, NSC719153, NSC720444, NSC721514, NSC726449, NSC727727, NSC727962, NSC728134, NSC8806 or NSC92498.

Mammals that can be treated with the methods and compositions of the invention include any domestic or zoo animal, and humans.

The methods and compositions of the invention can be used to treat or prevent a variety of cancers including carcinomas, adenocarcinomas, soft tumors and hard tumors. For example, the cancer can be a breast, bladder, colon, kidney, liver, lung, esophagus, gall bladder, ovary, pancreas, stomach, cervix, thyroid, prostate, skin, central nervous system or peripheral nervous system cancer. In some embodiments, the cancer is prostate cancer.

Another aspect of the invention is a method of treating or preventing cellular motility of a cell in a mammal that involves administering to the mammal an effective amount of an agent that increases the expression or activity of MxA in the mammal.

Another aspect of the invention is a method of treating or preventing cancer in a mammal by administering to the mammal a therapeutically effective amount of an MxA polypeptide.

Another aspect of the invention is a method of treating or preventing cancer in a mammal by administering to the mammal an effective amount of nucleic acid encoding an MxA polypeptide, wherein the MxA polypeptide is operably linked to a promoter that can effect expression of the MxA polypeptide. In some embodiments, the nucleic acid is administered locally. For example, the nucleic acid can be administered at the site of a tumor.

Another aspect of the invention is a method of improving survival of a mammal with cancer by administering an effective amount of an agent that can increase MxA expression.

Another aspect of the invention is a method of identifying an agent that inhibits metastatic cancer that involves contacting a cancer cell with a test agent, observing whether expression is increased from an MxA promoter within the cancer cell, and identifying a test agent that increases expression from the MxA promoter. The MxA promoter can be linked to a nucleic acid encoding a reporter molecule. For example, the reporter molecule can be luciferase.

Another aspect of the invention is a method of identifying an agent that inhibits metastatic cancer in a mammal comprising: (a) injecting the mammal with a tumor cell that comprises a first nucleic acid encoding a MxA promoter operably linked to a second nucleic segment encoding a reporter molecule; (b) administering a test agent to the mammal; and (c) observing whether tumor cells can be detected in the mammal at sites distance from the primary site of tumor cell injection; wherein the tumor cell can form a metastiatic tumor in the mammal. In some embodiments, the method can also include quantifying expression of the reporter molecule in tumor cells at the primary site of tumor cell injection or in tumor cells at sites distance from the primary site of tumor cell injection. The reporter molecule can be any conveniently detectable molecule, for example, luciferase.

### DESCRIPTION OF THE FIGURES

FIG. 1A-E illustrates the structure and expression of MxA. FIG. 1A provides a schematic diagram of the structure of the human MxA protein. Amino acid residues and locations of important motifs are indicated with shading and cross-hatching. In particular, the GTPase tripartite region is darkly shaded [symbol] , the self-assembling oligomerization domains that flank the GTPase region are identified by horizontal cross-hatching (≡), the dynamin-like regions are identified by slanted cross-hatching [symbol] and the two leucine zippers are identified by vertical cross-hatching (≡). One of the leucine zipper regions contains 4 spaced leucines (identified with “4”), while the other leucine zipper region contains 3 spaced leucines (identified with “3”). The asterisk identifies the site of the T103A mutation (Ponten et al., 1997). Two conservative sequence differences were found in MxA obtained from PC-3 cells and the homolog provided in the GenBank database. The first difference, I378V, resulted in a conservative amino acid change, while the second, at alanine 541 (GCA to GCG), was silent. There was no sequence alteration in the tripartite GTPase domain or the self-assembly domains. The difference may be the result of human sequence polymorphisms (Tazi-Ahnini et al., 2000).

FIG. 1B illustrates that MxA is expressed in PC-3 cells but not in PC-3M cells, as detected by Northern blot analysis. Ten μg of total RNA from these two prostate cancer cell lines were electrophoresed on a formaldehyde/agarose gel, blotted onto nylon membrane and probed as indicated with DD-2, MxA or GAPDH. The MxA probe used was an insert from the published MxA cDNA (Horisberger et al., 1990). The DD2 probe was an insert from the cloned 200-bp PCR fragment isolated by differential display of mRNA from PC-3. The GAPDH probe was the insert from rat glyceraldehyde dehydrogenase cDNA (Fort et al., 1985) and was used to control for equal loading. Sizes of bands shown are indicated on the left.

FIG. 1C illustrates that MxA is expressed in PC-3 cells but not in PC-3M cells, as detected by Western blot. Eighty (80) μg of cellular lysates were probed with affinity-purified goat anti-MxA antibody or mouse monoclonal and tubulin to control for equal loading. Sizes of bands are indicated on the left.

FIG. 1D illustrates that interferon-α (IFN-α) is expressed in PC-3 cells and in PC-3M cells, as detected by Western blot. One hundred (100) μg of cellular lysates were probed with a 1:1000 dilution of sheep anti-IFN-α globulin and sheep globulin lacking anti-IFN activity as control. Both antibody preparations were from the NIAID repository.

FIG. 1E illustrates that the genomic structure of PC-3 and PC-3M cells at the MxA locus is the same as detected by Southern Blot. Ten μg of genomic DNA from PC-3 and PC-3M were digested with EcoRI (RI), BamHI (B), or PstI (P), then separated by electrophoresis on a 1% agarose gel, blotted onto a nylon membrane and probed with insert from MxA cDNA. DNA size markers are indicated on the right.

FIG. 2A-B illustrates that prior to IFN-α treatment, the MxA protein was detected only in PC-3 cells and that after exposure to IFN-α the level of MxA protein increased substantially in PC-3. Mx-A protein expression was also induced in PC-3M cells. PC-3 (Figure A1-4) and PC-3M (FIG. 2B) cells were grown for 24 hours on cover slips in the presence or absence of 1000 international units of IFN-α ml¹. The cells were then fixed, permeabilized, stained with monoclonal anti-MxA and Cy-3-conjugated goat anti-mouse Ig antibody and counterstained with DAPI. Immunofluorescence was visualized with a Zeiss Axiophot microscope with a 40× objective, and the images were captured on an Optronics CCD camera.

FIG. 3A illustrates that PC-3M cells exhibit greater in vitro motility than PC-3 cells, and that IFN-α inhibits motility of both cell types. FIG. 3A graphically illustrates the motility of PC-3M and PC-3 cells, before and after exposure to 1000 IU ml⁻¹ IFN-α. Mean values of percent of control PC-3M mobility are shown as bar graphs with mean percentages indicated above each bar. Each value represents the mean of two independent triplicate determinations. Error bars show range of determinations.

FIG. 3B-C show that PC-3M cells transfected with Mx-A constructs exhibit less motility in a dose-dependent manner, where the amount of motility was inversely proportional to the level of Mx-A expression. FIG. 3B provides the level of Mx-A expression in two different PC-3M cell lines that were transfected with Mx-A expression cassettes. To assess levels of expression in these two cell lines, western blots of 50 μg of protein lysate per lane were probed with anti-MxA antibody. As illustrated, the MxA#4 line expressed more MxA than the MxA#4-2 line.

FIG. 3C graphically illustrates the motility of the MxA#4 and MxA#4-2 cell lines as compared to the PC-3M cells that express β-galactosidase. As illustrated, the MxA#4 cell line, which expresses more MxA, exhibited reduced levels of motility relative to the MxA#4-2 cells that express less MxA.

FIG. 4A-B illustrates that the invasiveness of cells correlates with the absence of wild type MxA activity in the highly metastatic melanoma cell line, LOX (Fodstad et al., 1988), which does not express endogenous MxA. LOX cells were transfected with expression cassettes encoding a FLAG-tagged wild-type MxA, a FLAG-tagged mutant (T103A) MxA that has inactive GTPase activity and that is unable to self-assemble (Ponten et al., 1997) or a FLAG-pCI-neocontrol. FIG. 4A shows the levels of MxA expression in these cell lines as observed by western blot of 50 μg of protein transfectant cell lysate per lane using anti-MxA antibodies as the probe. FIG. 4B illustrates the invasiveness of LOX cells that express wild-type MxA, the T103A mutant MxA or the FLAG-pCI-neo control. In FIG. 4B, the invasiveness is expressed as the percent of control cells (LOX cells with pCI neo vector alone) that successfully invaded the Matrigel clot and penetrated the PET membrane. The percentages are shown over each bar. The invasion assays on stable clone of LOX cells were done between 1 and 3 times, and a representative experiment is shown.

FIG. 5A-C shows that MxA is associated tubulin.

In FIG. 5A, PC-3 cell lysates (2.5 mg protein) were immunoprecipitated with either anti-α-tubulin or anti-actin antibodies and the bound proteins were detected by western blotting with anti-MxA antibody. Unprecipitated PC-3 lysate was also included on the western blot.

FIG. 5B shows that MxA GTPase activity is needed for association of MxA with tubulin. In FIG. 5B, cell lysates from LOX pCI neo, LOX FLAG-MxA WT and LOX FLAG-MxA T103A stably transfected cells (2.5 mg protein for all immunoprecipitations except 0.5 mg protein for immunoprecipitation with anti-MxA Ab) were immunoprecipitated with either beads alone, anti-α-tubulin or anti-MxA antibodies. The bound proteins were run on a western blot mat was probed with anti-FLAG antibody. Lane 3 is an empty lane.

FIG. 5C shows that MxA activity is needed for cytoskeletal localization of MxA—the MxA T103A mutant without GTPase activity does not associate with the cellular cytoskeleton. LOX cells that were stably transfected either with FLAG-tagged wild-type MxA or FLAG-tagged mutant MxA T103A were subjected to extraction procedures that left behind only insoluble cytoskeletal structures. The cells were then immunostained with anti-FLAG antibody. Nuclei of cells were visualized by counterstaining with DAPI.

FIG. 6A-B illustrates that MxA expression is correlated with slower tumor growth and improved survival of tumorous mice. In FIG. 6A, PC-3M β-gal and PC-3M MxA stably transfected cells were injected subcutaneously into beige/SCID mice, and the time to formation of a 2-cm subcutaneous mass was determined. As shown, 2-cm tumors took longer to form in mice receiving PC-3M MxA cells. In FIG. 6B, PC-3M-β-gal and PC-3M-MxA stably transfected cells were injected into the spleens of beige/SCID mice, and animal survival times were determined. Mean values and standard deviations are shown.

FIG. 7 outlines the high-throughput screen used for identifying small molecules that induce MxA expression. The MxA promoter was cloned upstream of luciferase, and transfected it into PC-3M human prostate carcinoma cells. A cell line that stably expressed this promoter-reporter construct was isolated. This cell line was used for testing whether test agents could induce MxA expression by screening for increased luciferase expression using a library of 1900 chemotypes. Interferon-α was a positive control for this assay.

FIG. 8 illustrates that certain compounds induce expression from the MxA promoter in PC-3M cells, including the NSC 34444, NSC 5159, NSC 46669, NSC 7215 and NSC 122335.

FIG. 9 illustrates that compounds NSC5159, NSC 46669, NSC 7215 and NSC 122335 induce expression of MxA protein.

FIG. 10 illustrates that compounds NSC 5159, NSC 46669, NSC 7215 and NSC 122335 decrease the motility of PC-3M cells.

FIG. 11 graphically illustrates which compounds induce expression from the MxA promoter in PC-3M cells. Induction of MxA promoter was assessed by observation of luciferase activity using the promoter-reporter construct described in FIG. 7. The x-axis lists compound numbers of compounds listed in Table 1.

FIG. 12A-C show structures of compounds that induce MxA promoter by two-fold or more.

FIG. 13 shows that MxA expression in PC-3-M tumor cells increases morbidity-free survival in mice. Mice were injected intrasplenically with PC-3-M cells that stably express neo-luciferase (PC-3-M-neo-luc), or with PC-3-M cells that stably express MxA-luciferase (PC-3-M-MxA-luc). Morbidity-free survival was assessed non-invasively over a period of 40 days using Xenogen technology.

### DETAILED DESCRIPTION OF THE INVENTION

As illustrated herein, compounds and methods that increase the expression of MxA decrease cellular motility and cancer cell metastasis, both in vitro and in vivo. Thus, the invention relates to compositions and methods for decreasing cell motility and inhibiting metastasis. The compositions and methods of the invention can be used to treat and prevent metastatic cancer.

Examples of compounds that can induce MxA expression and thereby reduce motility and metastasis of cancer cells include NSC 34444, NSC 122335, NSC 46669, NSC 7215, and NSC 5159. Other examples include NSC690269, NSC692406, NSC692407, NSC697537, NSC699152, NSC699167, NSC699491, NSC699782, NSC699881, NSC701744, NSC704660, NSC706453, NSC708444, NSC713080, NSC715435, NSC716204, NSC717200, NSC718885, NSC719153, NSC720444, NSC721514, NSC726449, NSC727727, NSC727962, NSC728134, NSC8806 and NSC92498.

Structures of compounds that can induce MxA expression and thereby reduce motility and metastasis of cancer cells have the following structures.

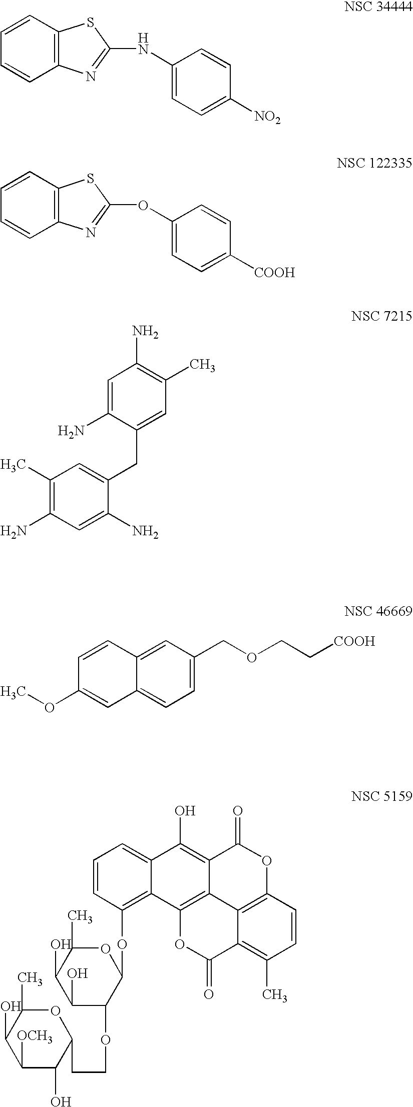

The NSC 34444 compound is benzothiazol-2-yl-(4-nitro-phenyl)-amine; the NSC 122335 compound is 4-(benzothiazol-2-yloxy)-benzoic acid; the NSC 46669 compound is 3-(6-Methoxy-naphthalen-2-ylmethoxy)-propionic acid; the NSC 7215 compound is methylene-bis(2,4-amino-5-methyl-benzene); the NSC 5159 compound is 10-{3-[2-(3,5-Dihydroxy-4-methoxy-6-methyl-tetrahydro-pyran-2-yl)-ethoxy]-4,5-dihydroxy-6-methyl-tetrahydro-pyran-2-yloxy}-6-hydroxy-1 methyl-benzo[h]chromeno[5,4,3-cde]chromene-5,12-dione.

Other agents that can modulate MxA expression include the following:

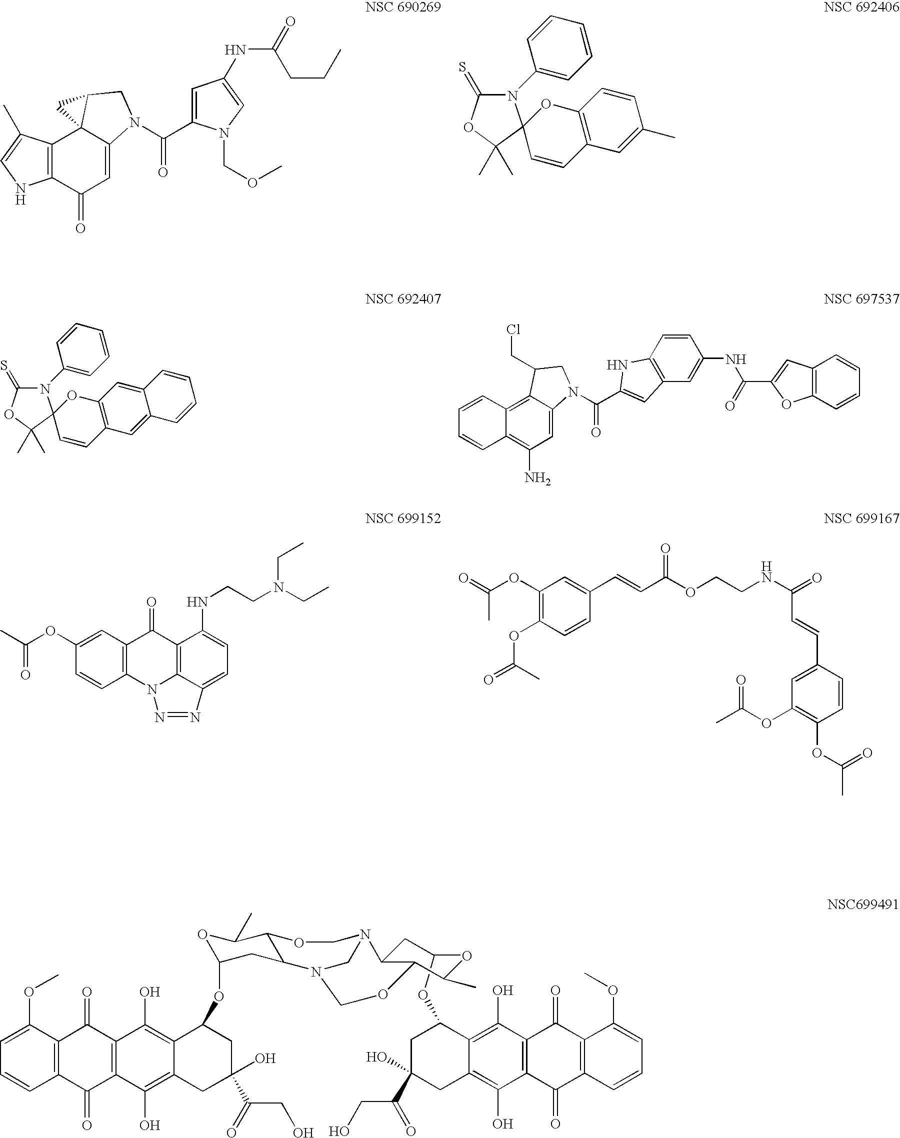

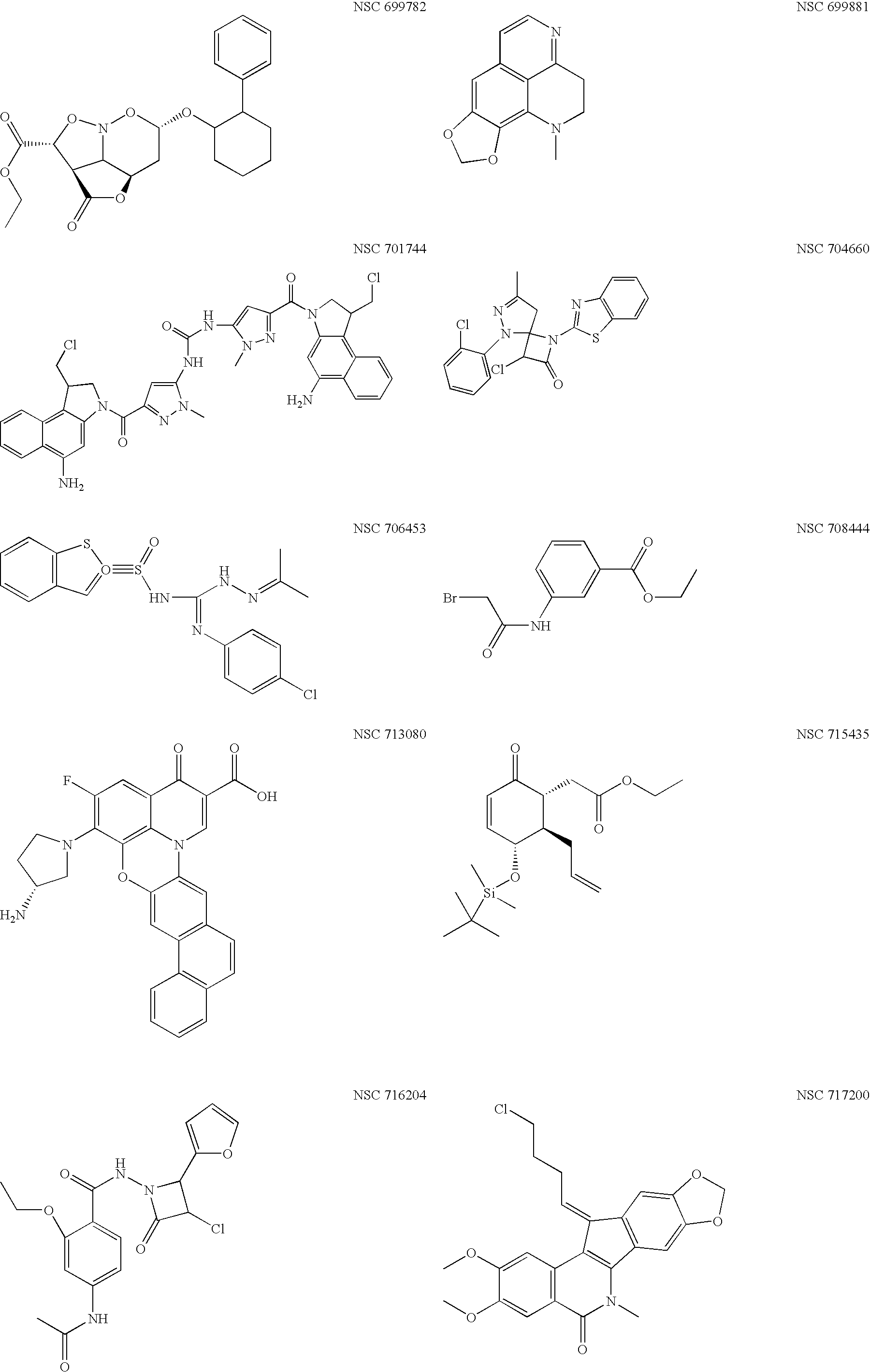

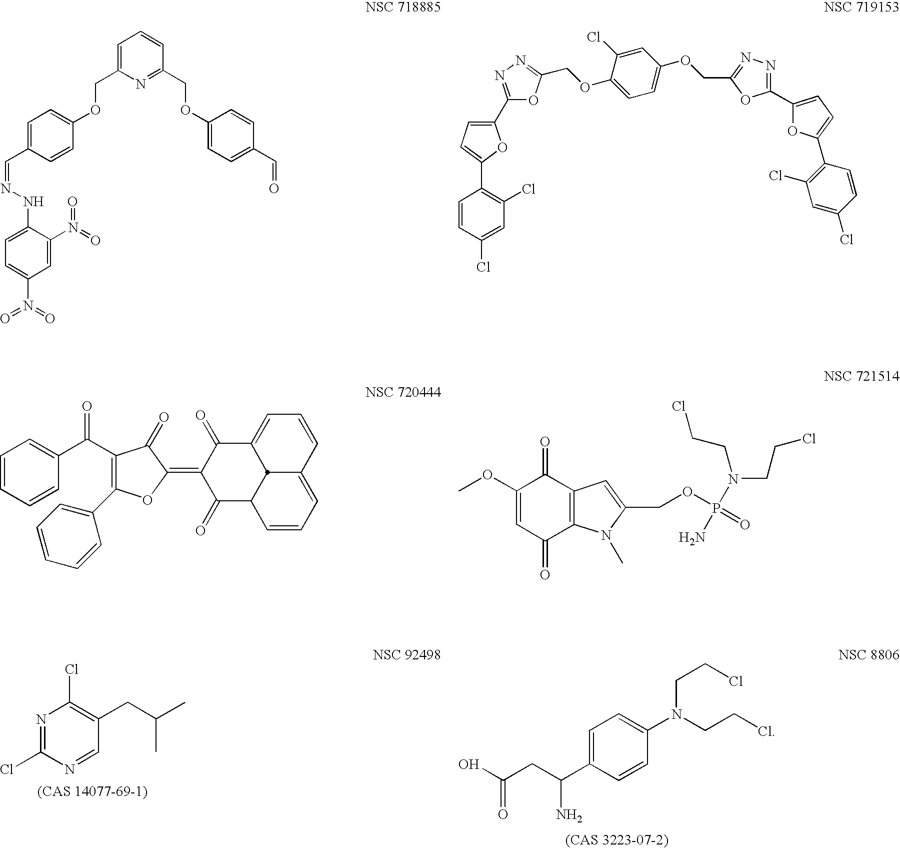

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or a fused bicyclic, tricyclic or quardrocyclic carbocyclic radical having about nine to twenty ring atoms in which at least one ring is aromatic. Heteroaryl encompasses a radical attached via a ring carbon of a monocyclic ring containing five to six ring atoms consisting of carbon and one to four heteroatoms each selected from the group consisting of non-peroxide oxygen, sulfur, and N(Y) wherein Y is absent or is H, O, (C₁-C₄)alkyl, phenyl or benzyl, as well as a radical of a fused bicyclic, tricyclic or quardrocyclic heterocycle of about eight to twenty ring atoms derived therefrom, particularly a benz-derivative or one derived by fusing a propylene, trimethylene, or tetramethylene diradical thereto.

Alkyl means (C₁-C₆)alkyl. Thus, for example, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “saccharide” includes monosaccharides, disaccharides, trisaccbarides and polysaccbarides. The term includes glucose, sucrose fructose and ribose, as well as deoxy sugars such as deoxyribose and the like. Saccharide derivatives can conveniently be prepared as described in International Patent Applications Publication Numbers WO 96/34005 and 97/03995. A saccharide can conveniently be linked to the remainder of a compound of formula II through an ether bond.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine cell migration or anti-viral activity using standard tests described herein, or using other similar tests that are well known in the art.

Specific and preferred values listed herein for radicals, substituents, and ranges, are for illustration only; they do not exclude other defined values or other values within defined ranges for the radicals and substituents.

Procedures available in the art can be used for synthesizing the compounds of the invention. For example, details on synthesizing organic compounds can be found in the art, for example, in Greene, T. W.; Wutz, P. G. M.

“Protecting Groups In Organic Synthesis” second edition, 1991, New York, John Wiley & sons, Inc.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### MxA

Differential display-reverse transcription-polymerization chain reaction (DD-RT-PCR) (Liang et al., 1992) was used to isolate mRNAs with expression differences in non-metastatic and metastatic cells. The non-metastatic tumor cells employed were PC-3 cells, derived from a prostate tumor and maintained as a prostate cancer cell line (Mickey, D. D., et al., “Characterization Of A Human Prostate Adenocarcinoma Cell Line (DU145) As A Monolayer Culture And As A Solid Tumor In Athymic Mice,” Prog. Clin. Biol. Res., 37:67-84 (1980), which is hereby incorporated by reference). A metastatic derivative of this cell line, the PC-3M cell line was used as a source of metastatic cells. The PC-3M cell line was derived from a liver metastasis in a nude mouse bearing a splenic explant of PC-3 (Kozlowski et al., 1984).

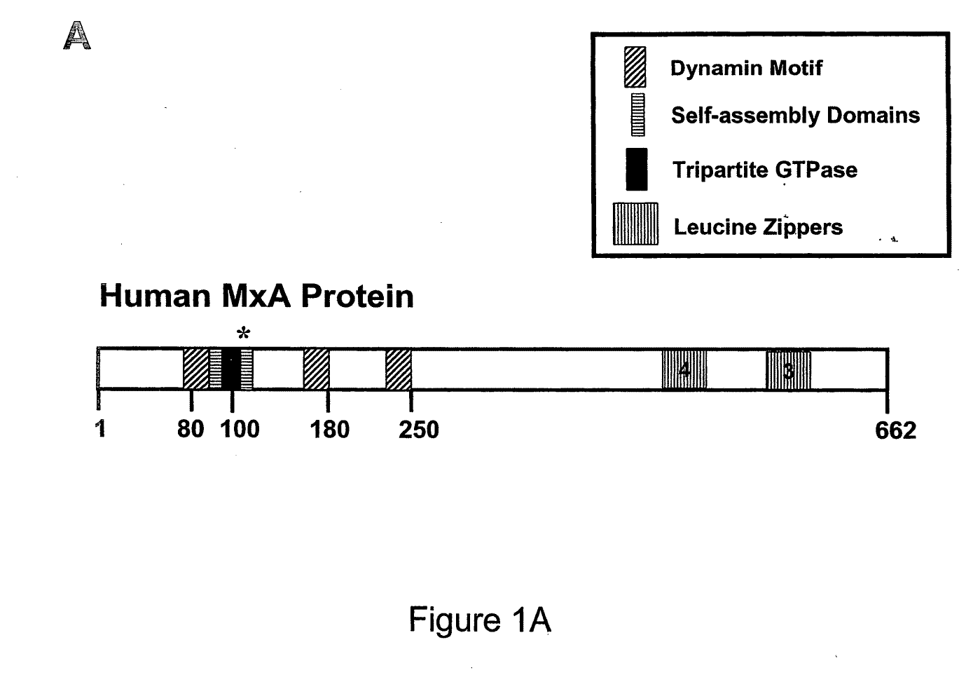

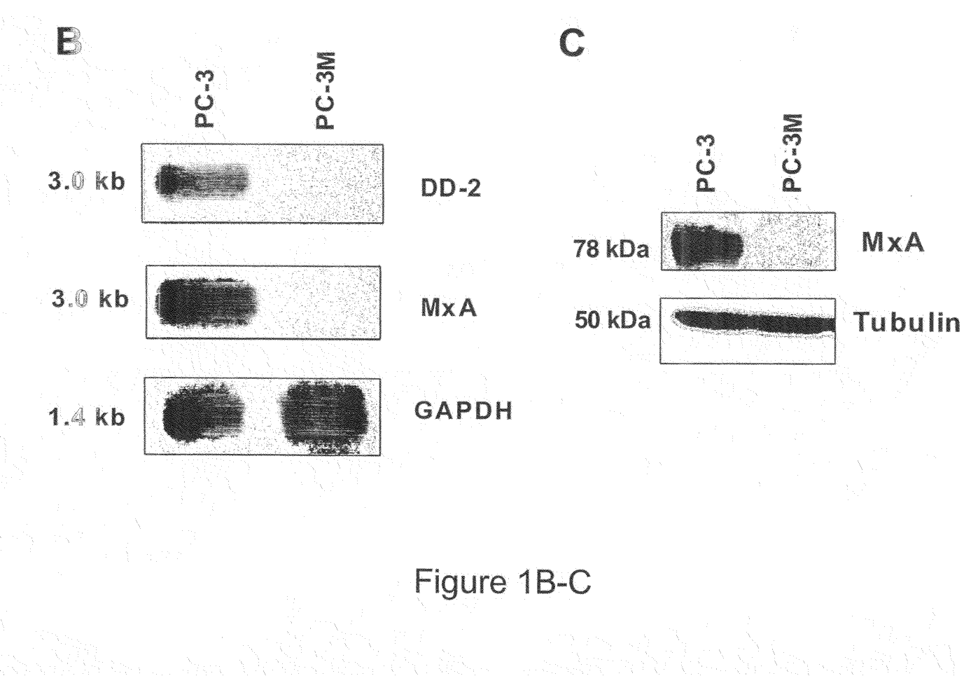

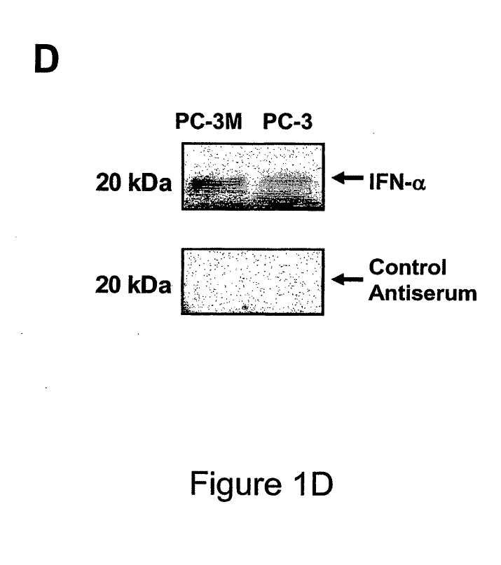

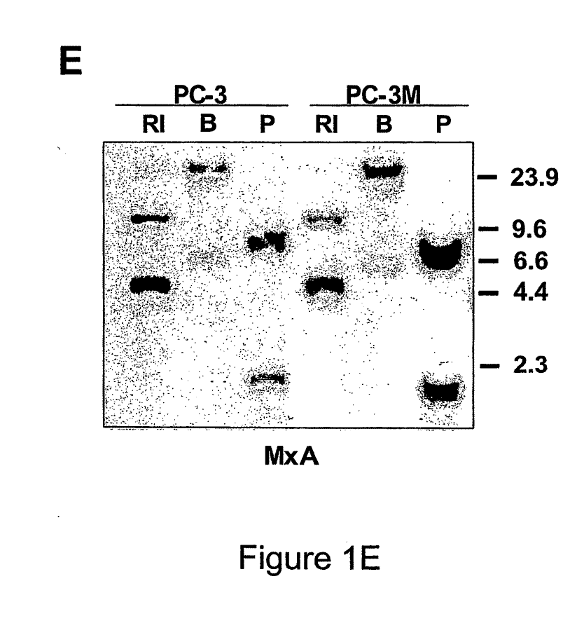

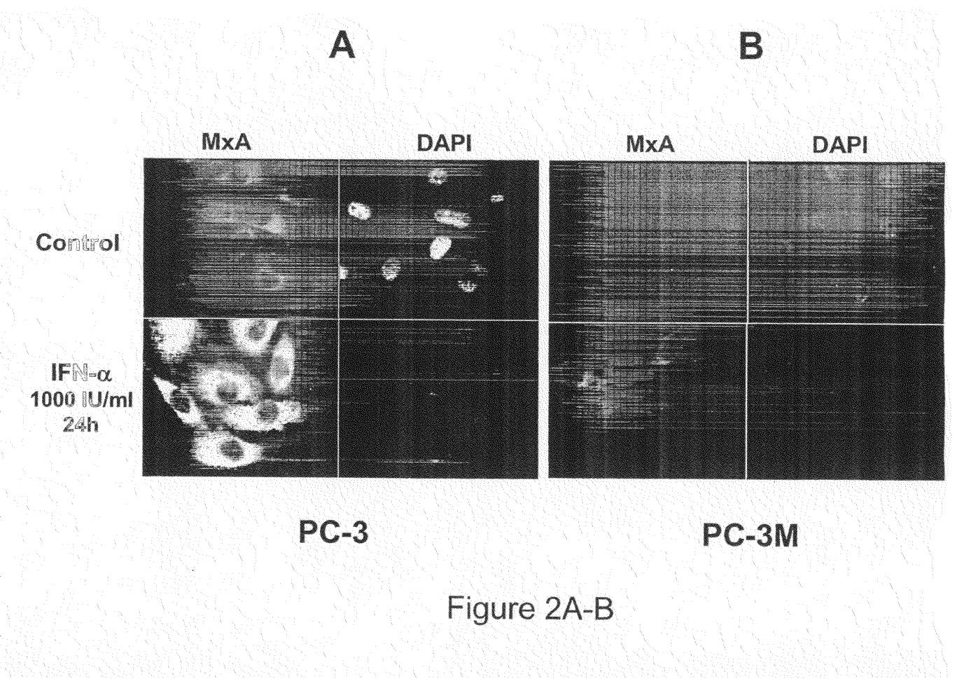

One clone isolated using this DD-RT-PCR assay was the DD-2 clone. As illustrated herein, PC-3M cells exhibit little or no expression of the DD-2 clone, compared to the PC-3 parent line. DNA sequencing of the DD-2 clone identified it as a portion of MxA, one of a small family of “Mx” genes (MxA and MxB are found in humans and Mx1 in mouse) that encode large self-assembling proteins that bind and hydrolyze GTP (Horisberger, 1992) (see FIG. 1A). One example of a sequence for an MxA polypeptide is as follows (SEQ ID NO:1).

| | |
|---|---|
| 1 | MVVSEVDIAK ADPAAASHPL LLNGDATVAQ KNPGSVAENN |
| 41 | LCSQYEEKVR PCIDLIDSLR ALGVEQDLAL PAIAVIGDQS |
| 81 | SGKSSVLEAL SGVALPRGSG IVTRCPLVLK LKKLVNEDKW |
| 121 | RGKVSYQDYE IEISDASEVE KEINKAQNAI AGEGMGISHE |
| 161 | LITLEISSRD VPDLTLIDLP GITRVAVGNQ PADIGYKIKT |
| 201 | LIKKYIQRQE TISLVVVPSN VDIATTEALS MAQEVDPEGD |
| 241 | RTIGILTKPD LVDKGTEDKV VDVVRNLVFH LKKGYMIVKC |
| 281 | RGQQEIQDQL SLSEALQREK IFFENHPYFR DLLEEGKATV |
| 321 | PCLAEKLTSE LITHICKSLP LLENQIKETH QRITEELQKY |
| 361 | GVDIPEDENE KMFFLIDKIN AFNQDITALM QGEETVGEED |
| 401 | IRLFTRLRHE FHKWSTIIEN NFQEGHKILS RKIQKFENQY |
| 441 | RGRELPGFVN YRTFETIVKQ QIKALEEPAV DMLHTVTDMV |
| 481 | RLAFTDVSIK NFEEFFNLHR TAKSKIEDIR AEQEREGEKL |
| 521 | IRLHFQMEQI VYCQDQVYRG ALQKVREKEL EEEKKKKSWD |
| 561 | FGAFQSSSAT DSSMEEIFQH LMAYHQEASK RISSHIPLII |
| 601 | QFFMLQTYGQ QLQKAMLQLL QDKDTYSWLL KERSDTSDKR |
| 641 | KFLKERLARL TQARRRLAQF PG |

Mx proteins have significant homology to dynamin, a molecular motor involved in coated vesicle-mediated endocytosis, and to VPSI, which is involved in intracellular protein trafficking (reviewed in Van der Bliek, 1999). However, heretofore, MxA has not been associated with cell motility or metastasis.

MxA protein is encoded by a nucleic acid having the tollowing sequence (SEQ ID NO:2).

| | |
|---|---|
| 1 | CCACGCGTCC GCCCAGTGTC ACGGTGGACA CGCCTCCCTC |
| 41 | GCGCCCTTGC CGCCCACCTG CTCACCCAGC TCAGGGGCTT |
| 81 | TGGAATTCTG TGGCCACACT GCGAGGAGAT CGGTTCTGGG |
| 121 | TCGGAGGCTA CAGGAAGACT CCCACTCCCT GAAATCTGGA |
| 161 | GTGAAGAACG CCGCCATCCA GCCACCATTC CAAGGAGGTG |
| 201 | CAGGAGAACA GCTCTGTGAT ACCATTTAAC TTGTTGACAT |
| 241 | TACTTTTATT TGAAGGAACG TATATTAGAG CTTACTTTGC |
| 281 | AAAGAAGGAA GATGGTTGTT TCCGAAGTGG ACATCGCAAA |
| 321 | AGCTGATCCA GCTGCTGCAT CCCACCCTCT ATTACTGAAT |
| 361 | GGAGATGCTA CTGTGGCCCA GAAAAATCCA GGCTCGGTGG |
| 401 | CTGAGAACAA CCTGTGCAGC CAGTATGAGG AGAAGGTGCG |
| 441 | CCCCTGCATC GACCTCATTG ACTCCCTGCG GGCTCTAGGT |
| 481 | GTGGAGCAGG ACCTGGCCCT GCCAGCCATC GCCGTCATCG |
| 521 | GGGACCAGAG CTCGGGCAAG AGCTCCGTGT TGGAGGCACT |
| 561 | GTCAGGAGTT GCCCTTCCCA GAGGCAGCGG GATCGTGACC |
| 601 | AGATGCCCGC TGGTGCTGAA ACTGAAGAAA CTTGTGAACG |
| 641 | AAGATAAGTG GAGAGGCAAG GTCAGTTACC AGGACTACGA |
| 681 | GATTGAGATT TCGGATGCTT CAGAGGTAGA AAAGGAAATT |
| 721 | AATAAAGCCC AGAATGCCAT CGCCGGGGAA GGAATGGGAA |
| 761 | TCAGTCATGA GCTAATCACC CTGGAGATCA GCTCCCGAGA |
| 801 | TGTCCCGGAT CTGACTCTAA TAGACCTTCC TGGCATAACC |
| 841 | AGAGTGGCTG TGGGCAATCA GCCTGCTGAC ATTGGGTATA |
| 881 | AGATCAAGAC ACTCATCAAG AAGTACATCC AGAGGCAGGA |
| 921 | GACAATCAGC CTGGTGGTGG TCCCCAGTAA TGTGGACATT |
| 961 | GCCACCACAG AGGCTCTCAG CATGGCCCAG GAGGTGGACC |
| 1001 | CCGAGGGAGA CAGGACCATC GGAATCTTGA CGAAGCCTGA |
| 1041 | TCTGGTGGAC AAAGGAACTG AAGACAAGGT TGTGGACGTG |
| 1081 | GTGCGGAACC TCGTGTTCCA CCTGAAGAAG GGTTACATGA |
| 1121 | TTGTCAAGTG CCGGGGCCAG CAGGAGATCC AGGACCAGCT |
| 1161 | GAGCCTGTCC GAAGCCCTGC AGAGAGAGAA GATCTTCTTT |
| 1201 | GAGAACCACC CATATTTCAG GGATCTGCTG GAGGAAGGAA |
| 1241 | AGGCCACGGT TCCCTGCCTG GCAGAAAAAC TTACCAGCGA |
| 1281 | GCTCATCACA CATATCTGTA AATCTCTGCC CCTGTTAGAA |
| 1321 | AATCAAATCA AGGAGACTCA CCAGAGAATA ACAGAGGAGC |
| 1361 | TACAAAAGTA TGGTGTCGAC ATACCGGAAG ACGAAAATGA |
| 1401 | AAAAATGTTC TTCCTGATAG ATAAAATTAA TGCCTTTAAT |
| 1441 | CAGGACATCA CTGCTCTCAT GCAAGGAGAG GAAACTGTAG |
| 1481 | GGGAGGAAGA CATTCGGCTG TTTACCAGAC TCCGACACGA |
| 1521 | GTTCCACAAA TGGAGTACAA TAATTGAAAA CAATTTTCAA |
| 1561 | GAAGGCCATA AAATTTTGAG TAGAAAAATC CAGAAATTTG |
| 1601 | AAAATCAGTA TCGTGGTAGA GAGCTGCCAG GCTTTGTGAA |
| 1641 | TTACAGGACA TTTGAGACAA TCGTGAAACA GCAAATCAAG |
| 1681 | GCACTGGAAG AGCCGGCTGT GGATATGCTA CACACCGTGA |
| 1721 | CGGATATGGT CCGGCTTGCT TTCACAGATG TTTCGATAAA |
| 1761 | AAATTTTGAA GAGTTTTTTA ACCTCCACAG AACCGCCAAG |
| 1801 | TCCAAAATTG AAGACATTAG AGCAGAACAA GAGAGAGAAG |
| 1841 | GTGAGAAGCT GATCCGCCTC CACTTCCAGA TGGAACAGAT |
| 1881 | TGTCTACTGC CAGGACCAGG TATACAGGGG TGCATTGCAG |
| 1921 | AAGGTCAGAG AGAAGGAGCT GGAAGAAGAA AAGAAGAAGA |
| 1961 | AATCCTGGGA TTTTGGGGCT TTCCAATCCA GCTCGGCAAC |
| 2001 | AGACTCTTCC ATGGAGGAGA TCTTTCAGCA CCTGATGGCC |
| 2041 | TATCACCAGG AGGCCAGCAA GCGCATCTCC AGCCACATCC |
| 2081 | CTTTGATCAT CCAGTTCTTC ATGCTCCAGA CGTACGGCCA |
| 2121 | GCAGCTTCAG AAGGCCATGC TGCAGCTCCT GCAGGACAAG |
| 2161 | GACACCTACA GCTGGCTCCT GAAGGAGCGG AGCGACACCA |
| 2201 | GCGACAAGCG GAAGTTCCTG AAGGAGCGGC TTGCACGGCT |
| 2241 | GACGCAGGCT CGGCGCCGGC TTGCCCAGTT CCCCGGTTAA |
| 2281 | CCACACTCTG TCCAGCCCCG TAGACGTGCA CGCACACTGT |
| 2321 | CTGCCCCCGT TCCCGGGTAG CCACTGGACT GACGACTTGA |
| 2361 | GTGCTCAGTA GTCAGACTGG ATAGTCCGTC TCTGCTTATC |
| 2401 | CGTTAGCCGT GGTGATTTAG CAGGAAGCTG TGAGAGCAGT |
| 2441 | TTGGTTTCTA GCATGAAGAC AGAGCCCCAC CCTCAGATGC |
| 2481 | ACATGAGCTG GCGGGATTGA AGGATGCTGT CTTCGTACTG |
| 2521 | GGAAAGGGAT TTTCAGCCCT CAGAATCGCT CCACCTTGCA |
| 2561 | GCTCTCCCCT TCTCTGTATT CCTAGAAACT GACACATGCT |
| 2601 | GAACATCACA GCTTATTTCC TCATTTTTAT AATGTCCCTT |
| 2641 | CACAAACCCA GTGTTTTAGG AGCATGAGTG CCGTGTGTGT |
| 2681 | GCGTCCTGTC GGAGCCCTGT CTCCTCTCTC TGTAATAAAC |
| 2721 | TCATTTCTAG CAGACAAAAA AAAAAAAAAA AAA |

MxA transcription is inducible by type-1 interferons (IFN) (Ronni et al., 1998), and MxA protein has been shown to be an effector of type I IFN-mediated inhibition of certain RNA viruses.

The sequence of the MxA promoter is provided below (SEQ ID NO:3).

| | |
|---|---|
| 1 | AAGCTTTATT ATTACTATTT TATTTATTTT ATTTTATTTT |
| 41 | CCTTCCACAC ACCCGTTTCC ACCCTGGAGA GGCCAGATGA |
| 81 | GCCAGACTCC AGGGAGGCCT AGAAGTGGGC AAGGGGAAAC |
| 121 | GGGAAAGGAG GAAGATGGTA TGGGTGTGCC TGGTTAGGGG |
| 161 | TGGGAGTGCT GGACGGAGTT CGGGACAAGA GGGGCTCTGC |
| 201 | AGCATTGCAC ACAATGCCTG GGAGTCCTGC TGGTGCTGGG |
| 241 | ATCATCCCAG TGAGCCCTGG GAGGGAACTG AAGACCCCCA |
| 281 | ATTACCAATG CATCTGTTTT CAAAACCGAC GGGGGGAAGG |
| 321 | ACATGCCTAG GTTCAAGGAT ACGTGCAGGC TTGGATGACT |
| 361 | CCGGGCCATT AGGGAGCCTC CGGAGCACCT TGATCCTCAG |
| 401 | ACTGGCCTGA TGAAACGAGC ATCTGATTCA GCAGGCCTGG |
| 441 | GTTCGGGCCC GAGAACCTGC GTCTCCCGCG AGTTCCCGCG |
| 481 | AGGCAAGTGC TGCAGGTGCG GGGCCAGGAG CTAGGTTTCG |
| 521 | TTTCTGCGCC CGGAGCCGCC CTCAGCACAG GGTCTGTGAG |
| 561 | TTTCATTTCT TCGCCGGCGC GGGCGGGGCT GGGCGCGCGG |
| 601 | TGAAAGAGGC GAACGAGAGC GGAGGCCGCA CTCCAGCACT |
| 641 | GCGCAGGGAC CGGTGAGTGT CGCTTCTGGG GGCAGCGCAG |
| 681 | TAACCGCGCT AGGAGCGCGA GAAGGGCATT GGGAGAGCGG |
| 721 | CGTTCGTGCG AGACTAGCGC TCCGGAGCAC GGGCACGACG |
| 761 | GGGGCACCTT CTCGGCTGCT AGTAACTAAC AATAATAATA |
| 801 | ATCATAATCA TAGCAAGGGC GCTGATGGGC GGGCTCGGAG |
| 841 | CACGCCTGAT TCTGGTTCCC ACCAGGCTGC CCAGGCTCCT |
| 881 | GATGACGCAT CAGAAACATC CCCCTAACCC GCGGCCTTCC |
| 921 | TGCAGGAGAG GTTGGGAAGG GGTGGGGGAC GGGGCTCGGG |
| 961 | GGAGGTCTCC GAGGGACTCT AGTAAGCGGG GAAGGGCGCC |
| 1001 | GGGAAAGTTT CAGATCCACG GTGCGCGGGC CACGAGCCAC |
| 1041 | CCGAACGCCG ACCACTGCTT TCCGTCGACT TCTATTTCCT |
| 1081 | GGGAACGCGC GAAACAAGAC CCAAGTCAGA CTGCGGAGGT |
| 1121 | CGCTGGGGAG GGAAGGTTCA AGGAGTTCTC GCCGATCCTG |
| 1161 | CTGAATAAAG GGGGTTCCGA GCTGGGCCGA GATGGGGCAT |
| 1201 | GCGCGGGAAG ACCCCTGCCC GCTGTTCCCC CCCACCGCCC |
| 1241 | CAGTGGATGC CATGCCTGGG GCTCCCCGGC GCGTGGGGCT |
| 1281 | GACGCACCCT CGGGTCCATC GTAGTTGGCC GGATCGTGGA |
| 1321 | GTGGGTGCGG TGGACGAAGG GAGGCAGGAC AGTCCCGGGG |
| 1361 | GTGGCAGAAG GAGCCCGGGC ACAGCTGAGA CCTGCGCTCC |
| 1401 | CATCCCACCA ACACTCACAG CAGGTGCTGC CGAGCTGGGC |
| 1441 | AATTGGGATG GCCCAAGTTA TTTGGTTAAA TTTTAAATCA |
| 1481 | CGTTTGTTAC TGGGAAGTAG AGTCCAGTGA TGCTAACCGC |
| 1521 | GCCTCTACCT CACCACCGGT GTCAGTCCAA AGGGCTCCTA |
| 1561 | AAATGGCTGT GTCATCTTTC AGCCTTGGAC CGCAGTTGCC |
| 1601 | GGCCAGGAAT CCCAGTGTCA CGGTGGACAC GCCTCCCTCG |
| 1641 | CGCCCTTGCC GCCCACCTGC TCACCCAGCT CAGGGGCTTT |
| 1681 | GGTAGGTAGC AGTGCATTTG GTCTAAAGGG CAAGATGTTC |
| 1721 | TCTCTTTTAT TCATAACAAA TTTAAATACC AGCAGGGTTT |
| 1761 | GGGGGGAAAA ACGCTTTCAG AAGAAAAGGT GAATGTCAGT |
| 1801 | CCTGCAAGAG TTAGTTTTAA AACTAGACTG AATTGGCACA |
| 1841 | TGTATACCTA TGTAACAAAC CTGCACGTTC TGCACATGTA |
| 1881 | CCCCAGAACT TAAAAGCTT |

### Treatment of Metastatic Cancer

As illustrated herein, expression of MxA reduces tumor growth and improves of the survival of mammals with tumors. Thus, the invention provides methods of treating or preventing metastatic cancer in a mammal that involve administering to the mammal a therapeutically effective amount of MxA. In another embodiment, the invention involves methods of treating or preventing metastatic cancer in a mammal that involves administering to the mammal a nucleic acid that encodes a MxA polypeptide, where the nucleic acid is operably linked to a nucleic acid encoding a promoter that can effect expression of the MxA polypeptide. In a further embodiment, the invention provides methods of treating or preventing metastatic cancer in a mammal that involve administering to the mammal a therapeutically effective amount of an agent that can increase endogenous MxA expression.

According to the invention, a variety of cancers can be treated or prevented including, but not limited to: carcinomas such as breast, bladder, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma. In some embodiments, the cancer is a carcinoma. In other embodiments, the cancer is an adenocarcinoma. In some embodiments, the cancer is prostate cancer.

### Treatment of Viral Infections

MxA is an effective anti-viral agent. For example, it has been shown that MxA provides all of the anti-influenza activity of interferon, and that MxA expression confers survival from an otherwise lethal dose of influenza. Thus, the invention contemplates using the compounds and other agents identified as described herein as anti-viral agents.

Therefore, one aspect of the invention is a method of treating viral infections in a mammal that involves administering to the mammal a therapeutically effective amount of an agent that can increase endogenous MxA expression.

The term “viral infection” refers to infection by agents capable of replicating in a host cell and includes infection by DNA and RNA viruses, viroids, prions. Viruses include both enveloped and non-enveloped viruses, for example, hepatitis A virus, hepatitis B virus, hepatitis C virus, human immunodeficiency virus (HIV), poxviruses, herpes viruses, adenoviruses, papovaviruses, parvoviruses, reoviruses, orbiviruses, picornaviruses, rotaviruses, alphaviruses, rubivirues, influenza virus type A and B, avian influenza (bird flu) and avian influenza A (H5N1), flaviviruses, coronaviruses, paramyxoviruses, morbilliviruses, pneumoviruses, rhabdoviruses, lyssaviruses, orthmyxoviruses, bunyaviruses, phleboviruses, nairoviruses, hepadnaviruses, arenaviruses, retroviruses, enteroviruses, rhinoviruses and the filovirus. Viruses also include, for example, hemorrhagic fever viruses (HFVs), Chikungunya virus, Japanese encephalitis virus, Monkey pox virus, variola virus, Congo-Crimean haemorrhagic fever virus, Junin virus, Omsk haemorrhagic fever virus, Venezuelan equine encephalitis virus, Dengue fever virus, Lassa fever virus, Rift valley fever virus, SARS coronavirus, Western equine encephalitis virus, Eastern equine encephalitis virus, Lymphocytic choriomeningitis virus, Russian Spring-Summer encephalitis virus, White pox, Ebola virus, Machupo virus, Smallpox virus, Yellow fever virus, Hantaan virus, Marburg virus, and Tick-borne encephalitis virus. A “viral organism” includes, but is not limited to, any of the above described viruses. Such viral organisms can be suspected or be capable of causing an infection in an animal.

### Identification of Anti-Migration Agents

As illustrated herein, agents that inhibit cell migration and/or tumor cell metastasis can be identified by observing whether those agents modulate the expression of the MxA promoter. Thus, one aspect of the invention is a method of identifying an agent that inhibits metastatic cancer that involves contacting a cancer cell with a test agent, observing whether expression is increased from an MxA promoter within the cancer cell, and identifying a test agent that increases expression from the MxA promoter. To permit easy detection, the MxA promoter can be operably linked to a nucleic acid encoding a reporter molecule. Any convenient reporter molecule can be used, For example, the reporter molecule can be β-galactosidase or luciferase.

Such assays can be performed in vitro or in vivo. One of skill in the art may choose first to observe the effects of test agents on expression from a MxA promoter using in vitro cell culture assays. After selection of agents that modulate MxA expression during such in vitro cell culture assays, one of skill in the art may then choose to perform an in vivo assay.

Thus, the invention is also directed to an in vivo method of identifying an agent that inhibits metastatic cancer in a mammal comprising: (a) injecting the mammal with a tumor cell that includes a first nucleic acid encoding a MxA promoter operably linked to a second nucleic segment encoding a reporter molecule; (b) administering a test agent to the mammal; and (c) observing whether tumor cells can be detected in the mammal at sites distance from the primary site of tumor cell injection; wherein the tumor cell can form a metastiatic tumor in the mammal. In some embodiments, the method can also include quantifying expression of the reporter molecule in tumor cells at the primary site of tumor cell injection or in tumor cells at sites distance from the primary site of tumor cell injection.

Any convenient cell line can be used for the in vitro assays. However, for in vivo testing, and in many embodiments for in vitro testing, the assay is performed by observing expression from the MxA promoter in a tumor cell. As illustrated herein, one convenient cell line is the human prostate carcinoma cell line PC-3. These PC-3 cells are available from the American Type Culture Collection (ATCC No. CRL-1435).

Delivery of MxA Polypeptides and/or Nucleic Acids

According to the invention, MxA polypeptides or nucleic acids can be administered to a mammal for a variety of reasons, including to decrease cell motility, treat viral infections, to identify anti-cancer agents or reduce cancer cell metastasis.

MxA nucleic acids can be used in expression cassettes or gene delivery vehicles, for the purpose of delivering an mRNA or oligonucleotide (with a sequence from a native mRNA or its complement), a full-length protein, a fusion protein, a polypeptide, a into a cell, preferably a eukaryotic or mammalian cell. According to the present invention, a gene delivery vehicle can be, for example, naked plasmid DNA, a viral expression vector comprising an MxA-encoding nucleic acid in conjunction with a liposome or a condensing agent.

MxA nucleic acids can be introduced into suitable host cells using a variety of techniques that are available in the art, such as transferrin-polycation-mediated DNA transfer, transfection with naked or encapsulated nucleic acids, liposome-mediated DNA transfer, intracellular transportation of DNA-coated latex beads, protoplast fusion, viral infection, electroporation and calcium phosphate-mediated transfection.

In one embodiment of the invention, the gene delivery vehicle comprises a promoter and an MxA nucleic acid. Promoters that can be used include inducible promoters, tissue-specific promoters and promoters that are activated by cellular proliferation, such as the thymidine kinase and thymidylate synthase promoters. Other promoters that can be used include promoters that are activated by infection with a virus, such as the α- and β-interferon promoters, and promoters that can be activated by a hormone, such as estrogen. Other promoters that can be used include the Moloney virus LTR, the CMV promoter, and the mouse albumin promoter.

A gene delivery vehicle can comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In some embodiments, the gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91102805).

Examples of retroviruses that can be utilized include avian leukosis virus (ATCC Nos. VR-535 and VR-247), bovine leukemia virus (VR-1315), murine leukemia virus (MLV), mink-cell focus-inducing virus (Koch et al., J. Vir. 49:828, 1984; and Oliff et al., J. Vir. 48:542, 1983), murine sarcoma virus (ATCC Nos. VR-844, 45010 and 45016), reticuloendotheliosis virus (ATCC Nos. VR-994, VR-770 and 45011), Rous sarcoma virus, Mason-Pfizer monkey virus, baboon endogenous virus, endogenous feline retrovirus (e.g., RD114), and mouse or rat gL30 sequences used as a retroviral vector. Strains of MLV from which recombinant retroviruses can be generated include 4070A and 1504A (Hartley and Rowe, J. Vir. 19:19, 1976), Abelson (ATCC No. VR-999), Freda (ATCC No. VR-245), Graffi (Ru et al., J. Vir. 67:4722, 1993; and Yantchev Neopksma 26:397, 1979), Gross (ATCC No. VR-590), Kirsten (Albino et al., J. Exp. Med. 164:1710, 1986), Harvey sarcoma virus (Manly et al., J. Vir. 62:3540, 1988; and Albino et al., J. Exp. Med. 164:1710, 1986) and Rauscher (ATCC No. VR-998), and Moloney MLV (ATCC No. VR-190). A non-mouse retrovirus that can be used is Rous sarcoma virus, for example, Bratislava (Manly et al., J. Vir. 62:3540, 1988; and Albino et al., J. Exp. Med. 164:1710, 1986), Bryan high titer (e.g., ATCC Nos. VR-334, VR-657, VR-726, VR-659, and VR-728), Bryan standard (ATCC No. VR-140), Carr-Zilber (Adgighitov et al., Neoplasma 27:159, 1980), Engelbreth-Holm (Laurent et al., Biochem Biophys Acta 908:241, 1987), Harris, Prague (e.g., ATCC Nos. VR-772, and 45033), or Schmidt-Ruppin (e.g. ATCC Nos. VR-724, VR-725, VR-354) viruses.

Any of the above retroviruses can be readily utilized in order to assemble or construct retroviral gene delivery vehicles given the disclosure provided herein and standard recombinant techniques (e.g., Sambrook et al., Molecular Cloning: A Laboratory Manual, 2^{nd} Edition (1989), Sambrook et al., Molecular Cloning: A Laboratory Manual, 3^{rd} Edition (2001), and Kunkle, Proc. Natl. Acad. Sci. U.S.A. 82:488, 1985). Portions of retroviral expression vectors can be derived from different retroviruses. For example, retrovector LTRs can be derived from a murine sarcoma virus, a tRNA binding site from a Rous sarcoma virus, a packaging signal from a murine leukemia virus, and an origin of second strand synthesis from an avian leukosis virus. These recombinant retroviral vectors can be used to generate transduction competent retroviral vector particles by introducing them into appropriate packaging cell lines (see Ser. No. 07/800,921, filed Nov. 29, 1991).

Recombinant retroviruses can be produced that direct the site-specific integration of the recombinant retroviral genome into specific regions of the host cell DNA. Such site-specific integration is useful for inserting MxA into convenient sites in the genome. Site-specific integration can be mediated by a chimeric integrase incorporated into the retroviral particle (see Ser. No. 08/445,466 filed May 22, 1995). It is preferable that the recombinant viral gene delivery vehicle is a replication-defective recombinant virus.

Packaging cell lines suitable for use with the above-described retroviral gene delivery vehicles can be readily prepared (see WO 92/05266) and used to create producer cell lines (also termed vector cell lines or “VCLs”) for production of recombinant viral particles. In some embodiments of the present invention, packaging cell lines are made from human (e.g., HT1080 cells) or mink parent cell lines, thereby allowing production of recombinant retroviral gene delivery vehicles that are capable of surviving inactivation in human serum. The construction of recombinant retroviral gene delivery vehicles is described in detail in WO 91/02505. These recombinant retroviral gene delivery vehicles can be used to generate transduction competent retroviral particles by introducing them into appropriate packaging cell lines. Similarly, adenovirus gene delivery vehicles can also be readily prepared and utilized given the disclosure provided herein (see also Berkner, Biotechniques 6:616-627, 1988, and Rosenfeld et al., Science 252:431-434, 1991, WO 93/07283, WO 93/06223, and WO 93/07282).

A gene delivery vehicle can also be a recombinant adenoviral gene delivery vehicle. Such vehicles can be readily prepared and utilized given the disclosure provided herein (see also Berkner, Biotechniques 6:616, 1988, and Rosenfeld et al., Science 252:431, 1991, WO 93/07283, WO 93/06223, and WO 93/07282). Adeno-associated viral gene delivery vehicles can also be constructed and used to deliver proteins or nucleic acids of the invention to cells in vitro or in vivo. The use of adeno-associated viral gene delivery vehicles in vitro is described in Chatteijee et al., Science 258: 1485-1488 (1992), Walsh et al., Proc. Nat'l. Acad. Sci. 89: 7257-7261 (1992), Walsh et al., J. Clin. Invest. 94: 1440-1448 (1994), Flotte et al., J. Biol. Chem. 268: 3781-3790 (1993), Ponnazhagan et al., J. Exp. Med. 179: 733-738 (1994), Miller et al., Proc. Nat'l Acad. Sci. 91: 10183-10187 (1994), Einerhand et al., Gene Ther. 2: 336-343 (1995), Luo et al., Exp. Hematol. 23: 1261-1267 (1995), and Zhou et al., Gene Therapy 3: 223-229 (1996). In vivo use of these vehicles is described in Flotte et al., Proc. Nat'l Acad. Sci. 90: 10613-10617 (1993), and Kaplitt et al., Nature Genet. 8:148-153 (1994).

In another embodiment of the invention, a gene delivery vehicle is derived from a togavirus. Such togaviruses include alphaviruses such as those described in U.S. Ser. No. 08/405,627, filed Mar. 15, 1995, WO 95/07994. Alpha viruses, including Sindbis and ELVS viruses can be gene delivery vehicles for nucleic acids of the invention. Alpha viruses are described in WO 94/21792, WO 92/10578 and WO 95/07994. Several different alphavirus gene delivery vehicle systems can be constructed and used to deliver nucleic acids to a cell according to the present invention. Representative examples of such systems include those described in U.S. Pat. Nos. 5,091,309 and 5,217,879. Preferred alphavirus gene delivery vehicles for use in the present invention include those that are described in WO 95/07994.

The recombinant viral vehicle can also be a recombinant alphavirus viral vehicle based on a Sindbis virus. Sindbis constructs, as well as numerous similar constructs, can be readily prepared. Sindbis viral gene delivery vehicles typically comprise a 5′ sequence capable of initiating Sindbis virus transcription, a nucleotide sequence encoding Sindbis non-structural proteins, a viral junction region inactivated so as to prevent fragment transcription, and a Sindbis RNA polymerase recognition sequence. Optionally, the viral junction region can be modified so that nucleic acid transcription is reduced, increased, or maintained. As will be appreciated by those in the art, corresponding regions from other alphaviruses can be used in place of those described above.

The viral junction region of an alphavirus-derived gene delivery vehicle can comprise a first viral junction region that has been inactivated in order to prevent transcription of the nucleic acid and a second viral junction region that has been modified such that nucleic acid transcription is reduced. An alphavirus-derived vehicle can also include a 5′ promoter capable of initiating synthesis of viral RNA from cDNA and a 3′ sequence that controls transcription termination.

Other recombinant togaviral gene delivery vehicles that can be utilized in the present invention include those derived from Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309 and 5,217,879 and in WO 92/10578.

Other viral gene delivery vehicles suitable for use in the present invention include, for example, those derived from poliovirus (Evans et al., Nature 339:385, 1989, and Sabin et al., J. Biol. Standardization 1:115, 1973) (ATCC VR-58); rhinovirus (Arnold et al., J. Cell. Biochem. L401, 1990) (ATCC VR-1110); pox viruses, such as canary pox virus or vaccinia virus (Fisher-Hoch et al., Proc. Natl. Acad. Sci. U.S.A. 86:317, 1989; FleK×ner et al., Ann. N.Y. Acad. Sci. 569:86, 1989; Flexner et al., Vaccine 8:17, 1990; U.S. Pat. Nos. 4,603,112 and 4,769,330; WO 89/01973) (ATCC VR-111; ATCC VR-2010); SV40 (Mulligan et al., Nature 277:108, 1979) (ATCC VR-305), (Madzak et al., J. Gen. Vir. 73:1533, 1992); influenza virus (Luytjes et al., Cell 59:1107, 1989; McMicheal et al., The New England Journal of Medicine 309:13, 1983; and Yap et al., Nature 273:238, 1978) (ATCC VR-797); parvovirus such as adeno-associated virus (Samulski et al., J. Vir. 63:3822, 1989, and Mendelson et al., Virology 166:154, 1988) (ATCC VR-645); herpes simplex virus (Kit et al., Adv. Exp. Med. Biol. 215:219, 1989) (ATCC VR-977; ATCC VR-260); Nature 277: 108, 1979); human immunodeficiency virus (EPO 386,882, Buchschacher et al., J. Vir. 66:2731, 1992); measles virus (EPO 440,219) (ATCC VR-24); A (ATCC VR-67; ATCC VR-1247), Aura (ATCC VR-368), Bebaru virus (ATCC VR-600; ATCC VR-1240), Cabassou (ATCC VR-922), Chikungunya virus (ATCC VR-64; ATCC VR-1241), Fort Morgan (ATCC VR-924), Getah virus (ATCC VR-369; ATCC VR-1243), Kyzylagach (ATCC VR-927), Mayaro (ATCC VR-66), Mucambo virus (ATCC VR-580; ATCC VR-1244), Ndumu (ATCC VR-371), Pixuna virus (ATCC VR-372; ATCC VR-1245), Tonate (ATCC VR-925), Triniti (ATCC VR-469), Una (ATCC VR-374), Whataroa (ATCC VR-926), Y-62-33 (ATCC VR-375), O'Nyong virus, Eastern encephalitis virus (ATCC VR-65; ATCC VR-1242), Western encephalitis virus (ATCC VR-70; ATCC VR-1251; ATCC VR-622; ATCC VR-1252), and coronavirus (Hamre et al., Proc. Soc. Exp. Biol. Med. 121:190, 1966) (ATCC VR-740).

A nucleic acid of the invention can also be combined with a condensing agent to form a gene delivery vehicle. In a preferred embodiment, the condensing agent is a polycation, such as polylysine, polyarginine, polyornithine, protamine, spermine, spermidine, and putrescine. Many suitable methods for making such linkages are known in the art (see, for example, Ser. No. 08/366,787, filed Dec. 30, 1994).

In an alternative embodiment, a nucleic acid is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell that has internalized the liposome, thereby releasing its contents into the cytoplasm. Prior to interaction with the surface of a cell, however, the liposome membrane acts as a relatively impermeable barrier that sequesters and protects its contents, for example, from degradative enzymes. Additionally, because a liposome is a synthetic structure, specially designed liposomes can be produced that incorporate desirable features. See Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., Proc. Natl. Acad. Sci. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising nucleic acids such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al, J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin™, from GIBCO BRL, Grand Island, N.Y. See also Feigner et al., Proc. Natl. Acad. Sci. US491: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatiyl ethanolamine (DOPE) and the like. These materials can also be mixed with the DOTMA and DOTAP starting materials in appropriate ratios. Methods for making liposomes using these materials are well known in the art.

The liposomes can comprise multilamellar vesicles (MLVs), small unilamellar vesicles (SUVs), or large unilamellar vesicles (LUVs). The various liposome-nucleic acid complexes are prepared using methods known in the art. See, e.g., Straubinger et al., Methods of Immunology (1983), Vol. 101, pp. 512-527; Szoka et al., Proc. Natl. Acad. Sci. USA 87:3410-3414, 1990; Papahadjopoulos et al., Biochim. Biophys. Acta 394:483, 1975; Wilson et al., Cell 17:77, 1979; Deamer and Bangham, Biochim. Biophys. Acta 443:629, 1976; Ostro et al., Biochem. Biophys. Res. Commun. 76:836, 1977; Fraley et al., Proc. Natl. Acad Sci. USA 76:3348, 1979; Enoch and Strittmatter, Proc. Natl. Acad Sci. USA 76:145, 1979; Fraley et al., J. Biol. Chem. 255:10431, 1980; Szoka and Papahadjopoulos, Proc. Natl. Acad. Sci. USA 75:145, 1979; and Schaefer-Ridder et al., Science 215:166, 1982.

In addition, lipoproteins can be included with a nucleic acid of the invention for delivery to a cell. Examples of such lipoproteins include chylomicrons, HDL, IDL, LDL, and VLDL. Mutants, fragments, or fusions of these proteins can also be used. Modifications of naturally occurring lipoproteins can also be used, such as acetylated LDL. These lipoproteins can target the delivery of nucleic acids to cells expressing lipoprotein receptors. Preferably, if lipoproteins are included with a nucleic acid, no other targeting ligand is included in the composition.

Receptor-mediated targeted delivery of MxA nucleic acids to specific tissues can also be used. Receptor-mediated DNA delivery techniques are described in, for example, Findeis et al. (1993), Trends in Biotechnol. 11, 202-05; Chiou et al. (1994), GENE THERAPEUTICS: METHODS AND APPLICATIONS OF DIRECT GENE TRANSFER (J. A. Wolff, ed.); Wu & Wu (1988), J. Biol. Chem. 263, 621-24; Wu et al. (1994), J. Biol. Chem. 269, 542-46; Zenke et al. (1990), Proc. Natl. Acad. Sci. U.S.A. 87, 3655-59; Wu et al. (1991), J. Biol. Chem. 266, 338-42.

In another embodiment, naked nucleic acid molecules are used as gene delivery vehicles, as described in WO 90/11092 and U.S. Pat. No. 5,580,859. Such gene delivery vehicles can be either DNA or RNA and, in certain embodiments, are linked to killed adenovirus. Curiel et al., Hum. Gene. Ther. 3:147-154, 1992. Other suitable vehicles include DNA-ligand (Wu et al., J. Biol. Chem. 264:16985-16987, 1989), lipid-DNA combinations (Feigner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

One can increase the efficiency of naked nucleic acid uptake into cells by coating the nucleic acids onto biodegradable latex beads. This approach takes advantage of the observation that latex beads, when incubated with cells in culture, are efficiently transported and concentrated in the perinuclear region of the cells. The beads will then be transported into cells when injected into muscle. Nucleic acid-coated latex beads will be efficiently transported into cells after endocytosis is initiated by the latex beads and thus increase gene transfer and expression efficiency. This method can be improved further by treating the beads to increase their hydrophobicity, thereby facilitating the disruption of the endosome and release of nucleic acids into the cytoplasm.

MxA nucleic acids can be introduced into cells in a similar manner. The nucleic acid construct encoding the MxA polypeptide may include transcriptional regulatory elements, such as a promoter element, an enhancer or UAS element, and a transcriptional terminator signal, for controlling transcription of the ribozyme in the cells. Mechanical methods, such as microinjection, liposome-mediated transfection, electroporation, or calcium phosphate precipitation, can be used to introduce the MxA construct into cells whose motility it is desired to decrease, as described above. Alternatively, if it is desired that the cells stably retain the construct, the construct can be supplied on a plasmid and maintained as a separate element or integrated into the genome of the cells, as is known in the art.

Expression of an endogenous MxA gene in a cell can also be altered by introducing in frame with the endogenous MxA gene a DNA construct comprising a MxA targeting sequence, a regulatory sequence, an exon, and an unpaired splice donor site by homologous recombination, such that a homologous recombinant cell comprising the DNA construct is formed. The new transcription unit can be used to turn the MxA gene on or off as desired. This method of affecting endogenous gene expression is taught in U.S. Pat. No. 5,641,670.

Integration of a delivered MxA nucleic acid into the genome of a cell line or tissue can be monitored by any means known in the art. For example, Southern blotting of the delivered MxA nucleic acid can be performed. A change in the size of the fragments of a delivered nucleic acid indicates integration. Replication of a delivered nucleic acid can be monitored inter alia by detecting incorporation of labeled nucleotides combined with hybridization to an MxA probe. Expression of an MxA nucleic acid can be monitored by detecting production of MxA mRNA that hybridizes to the delivered nucleic acid or by detecting MxA protein. MxA protein can be detected immunologically.

If one of skill in the art chooses to administer MxA polypeptides to a mammal, the entry of MxA polypeptides into cells can be facilitated by fusion of several protein transduction domains (PTDs) onto the MxA polypeptides. See Wadia & Dowdy (2002) Curr. Opin. Biotechnol. 13: 52-56. Such PTDs can transduce proteins across the plasma membrane, allowing the proteins to accumulate within the cell. The three most widely studied PTDs are from the Drosophila homeotic transcription protein antennapedia (Antp), the herpes simplex virus structural protein VP22 and the human immunodeficiency. virus 1 (HIV-1) transcriptional activator Tat protein. See, e.g., Joliet et al. (1991) Proc. Natl. Acad. Sci USA 88: 1864-68; Joliet et al. (1991) New Biol. 3: 1121-34; La Roux et al. (1993) Proc. Natl. Acad. Sci USA 90: 9120-24 (Drosophila Antp); Elliott & O'Hare (1997) Cell 88:223-33 (the herpes simplex virus VP22); Frankel & Pabo (1988) Cell 55: 1189-93.

Specific examples of PTDs that can be fused to the MxA polypeptides of the invention include, for example, any of the following:

| | | |
|---|---|---|
| Tat (43-60): | LGISYGRKKRRQRRRPPQ; | (SEQ ID NO: 4) |
| Tat (48-60): | GRKKRRQRRRPPQ; | (SEQ ID NO: 5) |
| Tat (47-57): | YGRKKRRQRRR; | (SEQ ID NO: 6) |
| and/or | | |
| Antp: | RQIKIWFQNRRMKWKK. | (SEQ ID NO: 7) |

These short peptide have the ability to internalize PTD-polypeptide fusions into cells and can also facilitate nuclear localization of the fusion proteins.

Transduction across the membrane by these PTDs occurs is independent of receptors, transporters and endocytosis. Moreover, transduction occurs via a rapid process at both 37° C. and 4° C., and essentially 100% or cells will take up the PTD-polypeptide fusion in a concentration-dependent fashion. Significantly, when synthesized as recombinant fusion proteins or covalently cross-linked to full-length proteins, these PTDs are capable of delivering biologically active proteins into mammalian cells. These PTD fusion proteins are found both within the cytoplasm and the nucleus.

Thus, the invention is directed to a PTD-MxA fusion protein that includes a MxA polypeptide (e.g. SEQ ID NO:1) and a PTD peptide (e.g., any one of SEQ ID NO:4-7).

### Compositions

The compounds and/or MxA polypeptides of the invention can be formulated as pharmaceutical compositions and administered to a mammal, such as a human patient in a variety of forms adapted to the chosen route of administration, i.e., orally or parenterally, by intravenous, intramuscular, topical or subcutaneous routes.

Thus, the present compounds and/or polypeptides may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compounds or polypeptides may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound(s) may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compounds and/or polypeptides in the required amount in the appropriate solvent with several of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds and/or polypeptides may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds and polypeptides of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compounds and/or polypeptides of the invention in a liquid composition, such as a lotion, will be from about 0.01-25 wt-%, preferably from about 0.1-10 wt-%. The concentration in a semi-solid or solid composition such as a gel or a powder will be about 0.01-10 wt-%, preferably about 0.1-5 wt-%.

The amount of the compound, polypeptide, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 1.0 to about 200 mg/kg, e.g., from about 2.0 to about 100 mg/kg of body weight per day, such as 5.0 to about 50 mg per kilogram body weight of the recipient per day, preferably in the range of 10 to 20 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 nM to about 10 μM, preferably, about 1 nM to 1 μM, most preferably, about 10 nM to about 0.5 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an inhibitor of cell migration or metastasis may be determined using pharmacological models that are well known to the art, or using the wound healing, chamber cell migration assay or tumor metastasis assays described below.

The Wound-Healing Assay involves observing whether confluent cells can migrate across a scrape or wound in the cell layer. For example, tumor cells can be plated in standard media containing 10% fetal bovine serum (FBS). After the cells grow to confluence, wounds are made in the confluent layer of cell using a sterile instrument such as a sterile pipette tip. The cells can be washed with Phosphate Buffered Saline (PBS) or other sterile solutions and then growth medium can be added that contains different concentrations of the compounds to be tested. After overnight incubation at 37° C., cells can be fixed and the plates can be photographed. Compounds and/or polypeptides that inhibit the migration of cells into the wound area at low concentrations are useful for inhibiting cell migration and treating metastatic cancer.

The Chamber Cell Migration Assay assesses whether cell can migrate through a filter having pores of known sizes. For example, cell migrations can be assayed with Boyden chambers having filters with about 8.0 μm pore size. Briefly, cells in serum-free medium are added to the first chamber and 500 μl of medium with 10% fetal bovine serum (FBS) is added to the second chamber. The chamber is incubated for about 6-8 hours at 37° C. with different concentrations of chemical compounds or polypeptides in both of the two chambers. Cells in the first chamber are removed with a cotton swab, and cells in the other chamber or on the other side of the filter are fixed and stained. Photographs several random regions of the filter facing the second chamber are taken and the number of cells counted to calculate the average number of cells that had transmigrated.

Moreover, the compounds, polypeptides and nucleic acids of the invention can be tested in appropriate animal models. For example, the compounds, polypeptides and nucleic acids of the invention can be tested in animals with known tumors, or animals that have been injected with tumor cells into a localized area. The degree or number of secondary tumors that form over time is a measure of metastasis and the ability of the compounds to inhibit such metastasis can be evaluated relative to control animals that have the primary tumor but receive no test compounds. Experimental results from this type of in vivo testing are described in the Examples. These results demonstrate that the compounds, nucleic acids and polypeptides of the invention substantially reduce or eliminate tumor metastasis.

Accordingly compounds, nucleic acids and polypeptides of the invention are useful as therapeutic agents for inhibition of cell migration and treatment of metastatic cancer. Such cancers include but are not limited to, cancers involving the animal's head, neck, lung, mesothelioma, mediastinum, esophagus, stomach, pancreas, hepatobiliary system, small intestine, colon, colorectal, rectum, anus, kidney, ureter, bladder, prostate, urethra, penis, testis, gynecological organs, ovaries, breast, endocrine system, skin, or central nervous system. Thus, for example, the cancer can be a breast cancer, a leukemia, a lung cancer, a colon cancer, a central nervous system cancer, a melanoma, an ovarian cancer, a renal cancer, or a prostate cancer.

Additionally, compounds of the invention may be useful as pharmacological tools for the further investigation of the inhibition of cell migration.

The compounds of the invention can also be administered in combination with other therapeutic agents that are effective for treating or controlling the spread cancerous cells or tumor cells.

The invention will now be illustrated by the following non-limiting Examples.

### Example 1

### MxA is Expressed in Non-Metastatic Tumor Cells But not in Metastatic Tumor Cells

This Example illustrates that MxA, a 78-kDa interferon-inducible GTPase, was expressed in the human prostate carcinoma cell line PC-3 but not in the highly metastatic derivative of these cells, the PC-3M cell line.

Constitutive expression of MxA in PC-3 cells. Differential display analysis using reverse transcriptase-polymerase chain reaction (RT-PCR) revealed eight cDNA fragments with possible differences in expression between PC-3 and its more metastatic derivative, PC-3M. Northern blot analysis indicated that only one of these, a 200-bp band, termed DD-2, was differentially expressed, as a strong 3.0-kb mRNA band in PC-3 but not in PC-3M (FIG. 1B). The DD-2 probe was used to screen a cDNA library generated from PC-3 mRNA, and a 2.0-kb cDNA clone was obtained that contained approximately 70% of the expected 3.0-kb sequence (including 95% of the coding region) of MxA, a 78-kDa IFN-inducible large GTPase. There was no significant difference in the inferred DD-2 cDNA amino acid sequence relative to the published inferred amino acid sequence of normal human embryonic lung MxA (Horisberger et al., 1990), which has an area of homology to the molecular motor dynamin, and two areas that enable self-assembly (FIG. 1A).

MxA expression has only been reported following viral infection or treatment with type 1 IFNs. However, northern blots probed with the original cDNA (Horisberger et al., 1990) displayed the same pattern of expression that was generated by the DD-2 probe: abundant expression in PC-3 but no detectible mRNA in PC-3M (FIG. 1B). Equal loading was determined by hybridizing the blots with a probe for glyceraldehyde phosphate dehydrogenase (GAPDH).

Western blot analysis with anti-MxA antibody (Horisberger and Hochkeppel, 1987) corroborated the northern blot expression data, demonstrating the presence of a 78-kDa MxA protein in PC-3 but not in PC-3M lysates (FIG. 1C). The same blot was probed with anti-tubulin antibody to show that both samples were equally loaded.

The difference between the cell lines could have been related to a high endogenous production of type-I IFN in PC-3 cells. This possibility was tested and there was, however, no difference in synthesis of IFN-α in the two cell lines (FIG. 1D).

Maintenance of genomic integrity at the MxA locus. The constitutive expression of MxA in PC-3 cells, but not in PC-3M cells, could be explained by a genomic deletion or rearrangement at the MxA locus. To explore this possibility, genomic DNAs from PC-3 and PC-3M cells were digested with EcoRI, Bam H1 and Pst1, electrophoresed on an agarose gel and subjected to Southern blot analysis with MxA cDNA (FIG. 1 E). PC-3 and PC-3M showed identical patterns of hybridization, which indicated that the difference in expression of MxA in PC-3 cells and PC-3M cells was not due to a major genomic deletion or rearrangement.

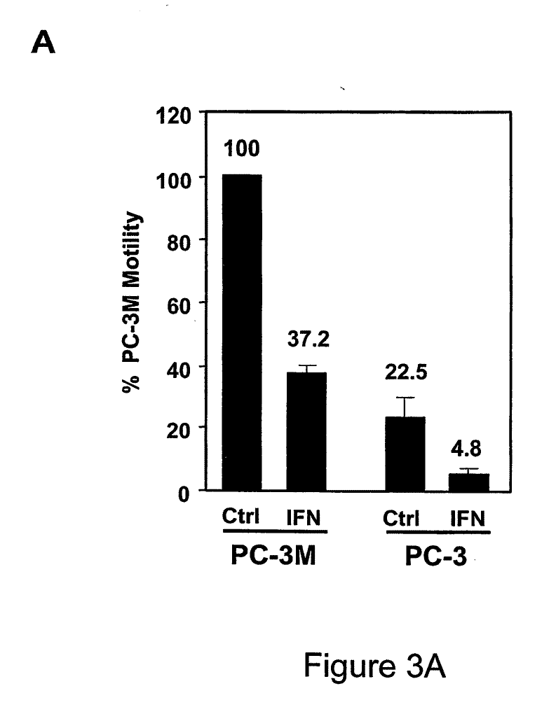

Induction of MxA expression by interferon. To determine whether IFN-α can induce MxA expression in PC-3 and PC-3M cells, cells were treated with recombinant IFN-α and subjected to immunohistochemical analysis using anti-MxA antibody and DAPI nuclear counterstaining to locate individual cells (FIG. 2). Consistent with the western blot result, this assay detected MxA protein only in untreated PC-3 and not in untreated PC-3M cells (compare upper left panels in FIGS. 2A and 2B). After exposure to IFN-α, the level of MxA protein increased substantially in PC-3, while MxA protein became detectable for the first time in PC-3M cells (compare lower left panels in FIGS. 2A and 2B). Western blotting (not shown) confirmed the IFN did induce increased MxA protein expression in both cell lines. This evidence indicated that PC-3M cells were still able to respond to interferon, consistent with the Southern blot result (FIG. 1E) that indicated that the MxA gene was intact in PC-3M cells. This experiment also showed that the IFN signaling pathway was still active in PC-3M cells, ruling out the possibility that lack of MxA expression in PC-3M cells was due to an inability to respond to IFN stimulation.

### Example 2

### MxA Inhibits Cell Tumor Cell Motility

This Example illustrates that increased MxA expression inhibits tumor cell motility.

Effect of MxA on motility of PC-3M. The constitutive expression of MxA in PC-3 cells and the absence of expression in PC-3M cells suggested that MxA might suppress some aspect of metastatic behavior. It has been reported that type I IFN could reduce cell motility (Brouty-Boyé and Zetter, 1980), one component of metastasis. To test whether PC-3 and PC-3M cells differed in motility, these two cell lines were subjected to a cell migration assay that measured the ability of cells to migrate through pores in a polyethylene terephthalate (PET) membrane.

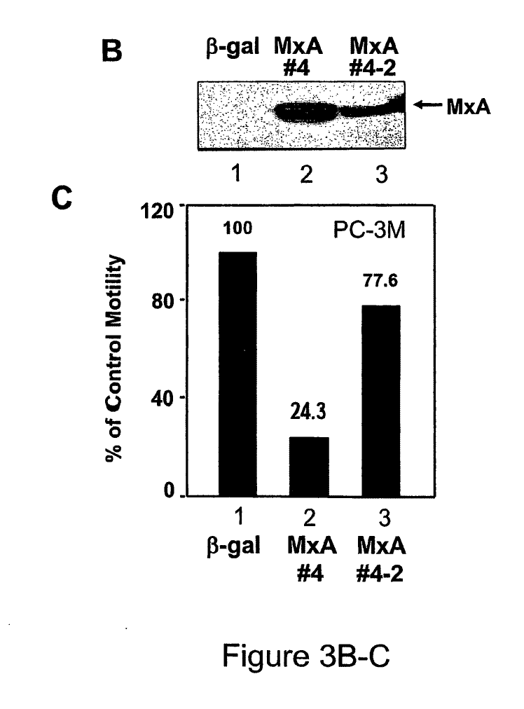

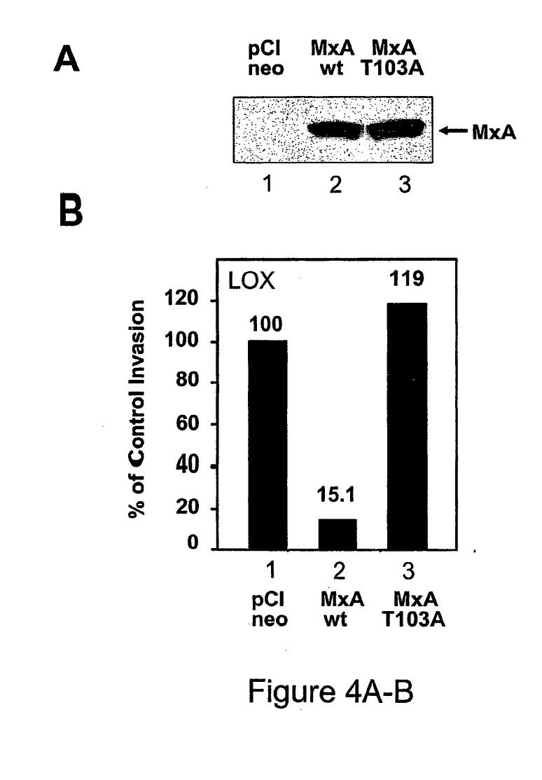

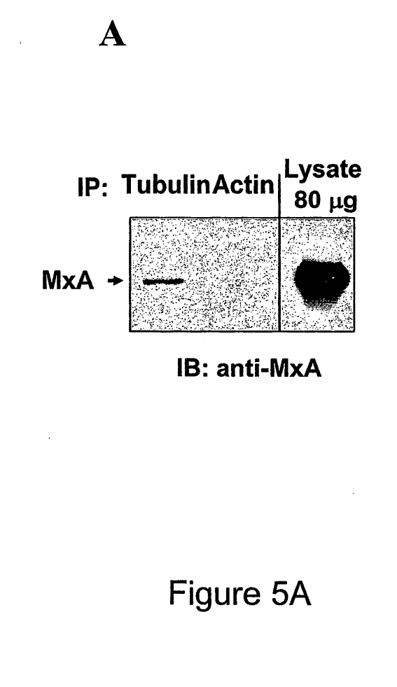

FIG. 3A shows that PC-3M cells were considerably more motile than PC-3 cells, and that IFN-α reduced PC-3M cell motility to a level comparable to that of PC-3 cells. Consistent with the results seen in FIG. 2, PC-3 was responsive to IFN, which reduced its motility to levels below that seen when no IFN was present.

To test whether MxA played a role in regulating motility, PC-3M cells were transfected with vectors that express MxA and β-galactosidase (control) (see Horisberger, 1995). Stable cell lines were selected including PC-3M-MxA#4 and PC-3M-MxA#4-2 cell lines, which constitutively expressed full-length human MxA protein, and a control cell line, PC-3M β-gal. The level of MxA expression in the three cell lines was determined by western blot (FIG. 3B). MxA protein was not detected in PC-3M β-gal cells, while the other two cells lines expressed exogenously introduced MxA. The MxA#4 cells expressed higher levels of MxA than the MxA#4-2 cells. MxA expression markedly inhibited motility in both clones (FIG. 3C), and the level of inhibition correlated well with the level of MxA expression.

Time-lapse microscopy. Time-lapse video microscopy further confirmed that MxA expression in PC-3M cells visually reduced cellular motility (data not shown). PC-3M-MxA#4 cells that had been stably transfected with MxA showed markedly reduced levels of movement across the field, compared to control PC-3M cells that expressed the unrelated protein, β-galactosidase. Both motion pictures showed active movement of plasma membrane in most cells and several cell divisions, indicating that over-expression of neither MxA nor β-galactosidase interfered with mitosis or membrane ruffling.

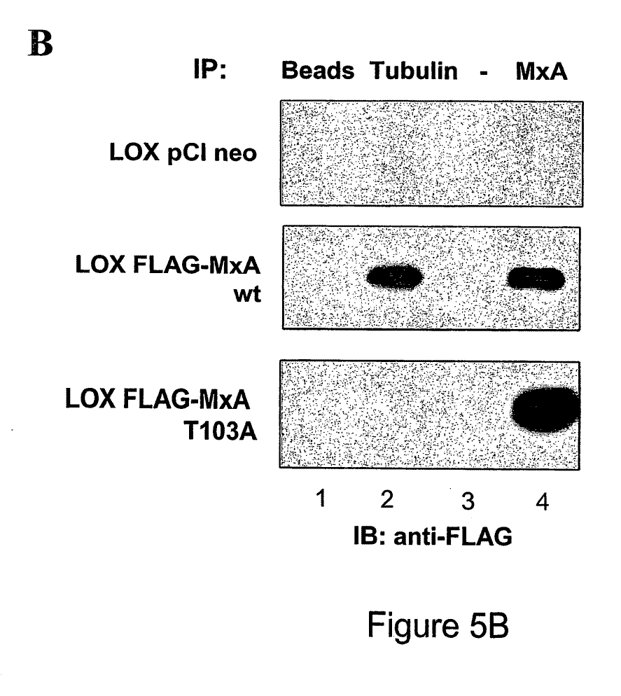

Effect of MxA expression on motility and invasiveness of the melanoma cell line, LOX. To determine if MxA would also inhibit motility of other tumor cell types, these studies were repeated using the highly metastatic melanoma cell line, LOX (Fodstad et al., 1988), which does not express endogenous MxA. Using transfection with a FLAG expression vector, stable LOX derivatives were created that expressed a FLAG-tagged wild-type MxA, a FLAG-tagged mutant (T103A) MxA that has inactive GTPase activity and that is unable to self-assemble (Ponten et al., 1997) and a FLAG-pCI-neo control. Cells expressing these constructs were tested in the same in vitro motility assay employed for generating the results in FIG. 3C. MxA was expressed at similar levels in both wild type and mutant transfectants, but not in cells transfected with vector alone (FIG. 4A). The expression of exogenous MxA in LOX cells also decreased their motility to a degree similar (data not shown) to that seen for PC-3M in FIG. 3C.

The LOX transfectants were also tested in an in vitro invasion assay. In this assay, cells were required to make their way through a Matrigel clot before they encountered the PET membrane and migrated through its pores. The results of the invasion assay are shown in the bar graph of FIG. 4B, which illustrates that expression of wild-type MxA (bar 2) significantly inhibited the in vitro invasive activity of LOX cells, compared with vector-alone controls (bar 1). However, the T103A mutation, in the dynamin/self-assembly region, completely reversed the ability of MxA to suppress in vitro invasiveness of LOX cells (FIG. 4B, bar 3).

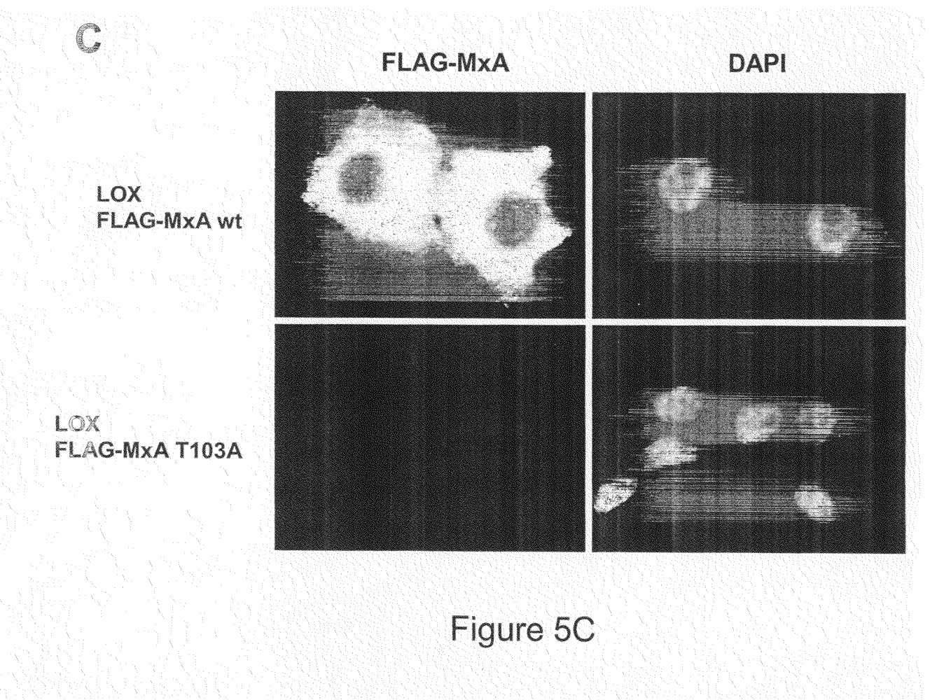

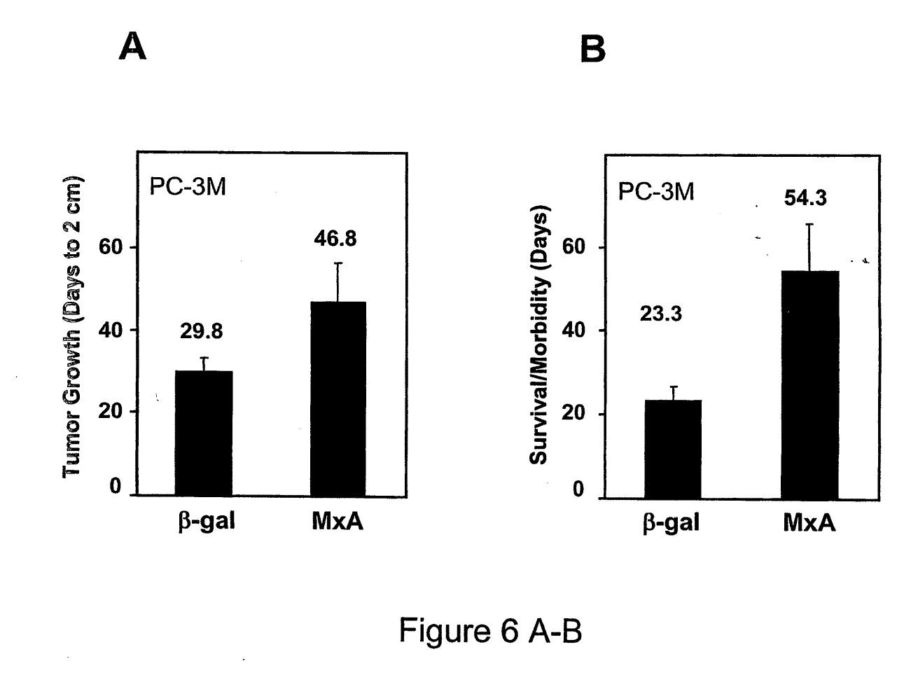

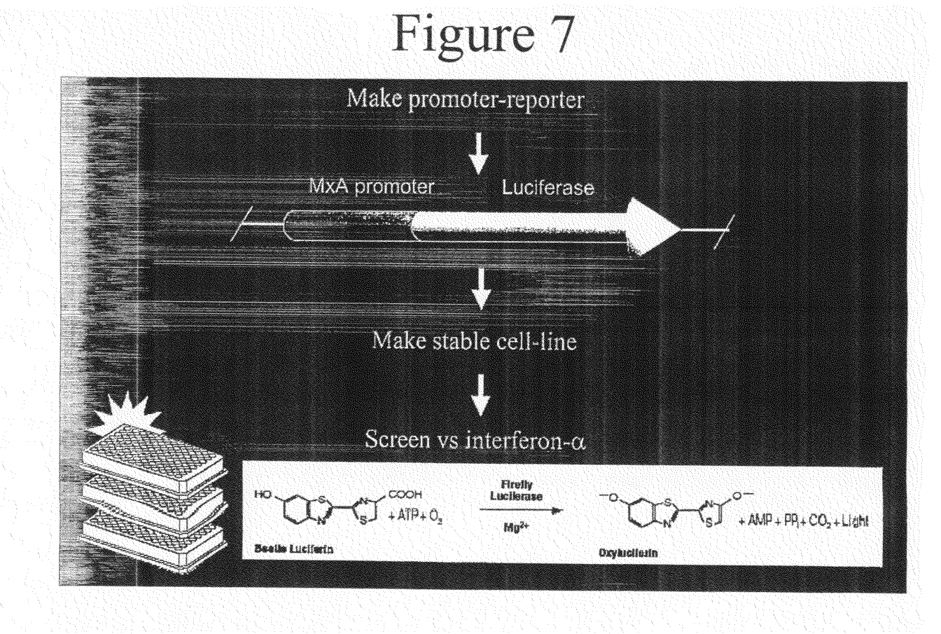

Wild-type MxA but not mutant MxA associates with tubulin. It has been reported previously that MxA can transiently bind elements of the cytoskeleton such as actin and tubulin (Horisberger, 1992). Because elements of the cytoskeleton are instrumental in cell motility, tests were performed to ascertain whether MxA was associated with the actin or tubulin cytoskeleton in PC-3 and LOX cells. These tests involved using immunoprecipitation and immunohistochemical studies of cytoskeleton preparations. FIG. 5A demonstrates that endogenous MxA co-immunoprecipitated with tubulin, but not with actin, in PC-3 cells.

To confirm that wild-type MxA associated with microtubules is a general phenomenon, a coimmunoprecipitation experiment was performed using other cell lines—the stably transfected LOX cell lines. Whole cell lysates were immunoprecipitated with anti-α-tubulin, anti-MxA antibodies or protein A/G-coated Sepharose beads alone, followed by western blotting with anti-FLAG antibody (FIG. 5B). As expected, MxA was detected in the complex with tubulin in LOX-FLAG-MxA WT (FIG. 5B, lane 2) while protein A/G alone (FIG. 5B, lane 1) did not bind MxA-containing complexes. No binding activity was detected in LOX-pCT-neo control cells (FIG. 5B, lane 1), indicating that the co-immunoprecipitation was specific for FLAG-tagged MxA constructs.

To test whether the association of MxA with the microtubule cytoskeleton was dependent upon its GTPase/self-assembly activity, as was MxA's ability to suppress motility and invasion, co-immunoprecipitation experiments were also performed using the LOX-T103A MxA stable lines (FIG. 5B, lane 4). In contrast to the LOX cells that expressed wild-type MxA, in LOX-FLAG-MxA T103A cells, the binding of the T103A mutant of MxA to tubulin was virtually undetectable.

When all soluble proteins were extracted from LOX melanoma cells that stably expressed wild-type MxA or T103A mutant MxA, only wild-type MxA protein remained bound to the insoluble cytoskeletal matrix (FIG. 5C). These data are consistent with the co-immunoprecipitation experiments in FIGS. 5A and 5B that showed that only wild-type MxA associated with tubulin. T103A MxA washed out of the insoluble cytoskeleton preparation, indicating that the mutant MxA protein is soluble and not bound to any cytoskeletal elements (FIG. 5C).

These data indicate that an association exists between tubulin and wild-type MxA but not between tubulin and mutant MxA, suggesting that microtubules play a role in the MxA-mediated reduction of motility, invasiveness and metastasis of PC-3M prostate cancer cells and LOX melanoma cells.

### Example 3

### MxA Inhibits Cell Tumor Metastasis In Vivo

This Example illustrates that MxA expression

Effects of MxA on in vivo tumor growth and metastasis. The effect of MxA expression on tumor growth in vivo was tested using two experimental animal assays. In particular, a primary tumor growth assay and an experimental hepatic metastasis assay were used to assess tumor growth in vivo. In the first assay, 2×10⁶ PC-3M-MxA or 2×10⁶ PC-3M-β-gal cells were injected subcutaneously into 30 beige/SCID mice, and the time to formation of a 2-cm subcutaneous mass was determined.

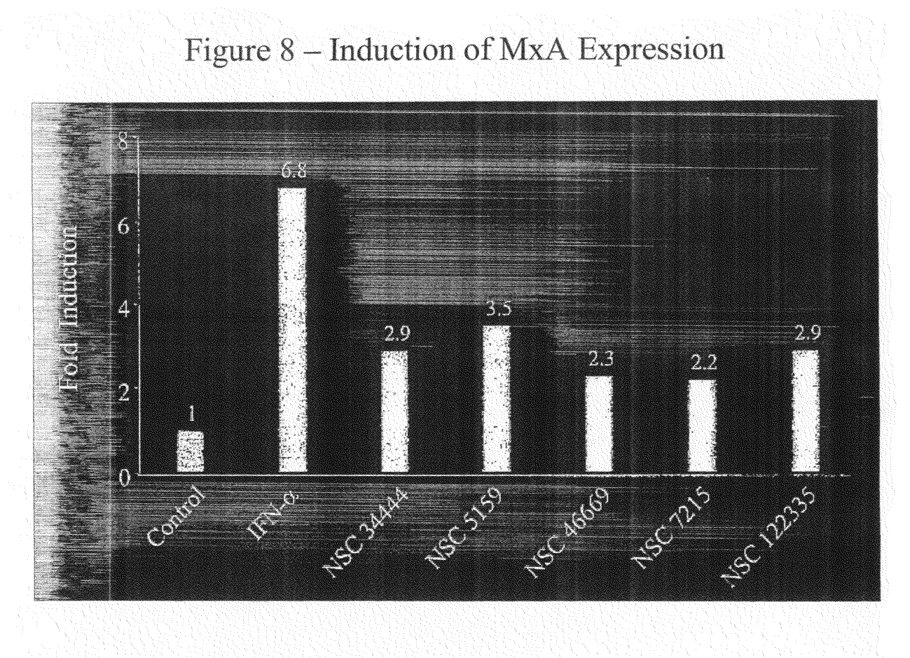

The subcutaneous tumorigenicity of PC-3M-β-gal and PC-3M-MxA cell lines was similar and high (14/15 and 15/15 respectively). Subcutaneous tumor growth (FIG. 6A) occurred significantly earlier (p<0.001) in PC-3M-β-gal (29.8±3.4 days) than in PC-3M-MxA (46.8±9.9 days). At the time of sacrifice, metastases from subcutaneous tumors were seen in both PC-3M-β-gal- and PC-3M-MxA-engrafted mice. Sites of metastases were similar for both cell lines, including regional lymph nodes and lung.

To assess the effect of MxA on metastatic potential, a hepatic metastasis assay was employed. In this assay, 2×10⁶ cells from the same two cell lines were injected into the spleens of beige/SCID mice. The primary endpoint of this assay was survival (hepatic metastasis-associated morbidity). There were liver metastases detected in both groups at the time of death or sacrifice. The metastases of PC-3M-β-gal cells occurred earlier and resulted in more rapid metastasis-associated morbidity than PC-3M-MxA cells (FIG. 6B). In addition, there was a markedly greater replacement of normal liver parenchyma by the PC-3M-β-gal metastases. The survival time of the PC-3M-MxA-engrafted mice (54.3±11.2 days) was significantly longer (p<0.001)—twice that of the PC-3M-β-gal-engrafted mice (23.3±3.3 days). All mice had solitary splenic tumor nodules at the site of injection that did not appear to contribute to morbidity. These animal data demonstrate that MxA slows the development of experimental metastases, and mitigates certain aspects of tumorigenesis in vivo.

As demonstrated here, MxA mRNA and protein were abundant in PC-3 but were not detectable in its more metastatic derivative, PC-3M. To test the hypothesis that MxA plays a role in reduction of motility and metastasis, we expressed the full-length MxA cDNA in PC-3M prostate carcinoma cells and in LOX melanoma cells and compared its effect with control vectors. MxA induced a clear reduction in motility and invasion in both tumor types in two in vitro assays. Stable expression of exogenous MxA in PC-3M cells also caused a significant reduction in two in vivo assays of malignancy in immunocompromised beige-SCID mice: growth rate of subcutaneous tumors and mortality from hepatic metastasis of splenic xenografts. These data demonstrate a role for MxA as an inhibitor of tumor cell metastasis.

It was unexpected that PC-3 cells express MxA spontaneously, since it is believed that MxA is not expressed in normal or neoplastic cells in the absence of viral infection or exposure to exogenous interferon (Goetschy et al., 1989; al-Masri et al., 1997). However, western blot analysis of 27 cancer cell lines of the NCI 60 panel (Scherf et al., 2000) detected MxA expression in 0/1 leukemia, 3/7 non-small cell lung cancer, 2/7 colon, 4/6 CNS and 2/6 melanoma (data not shown). This suggests that the regulation of MxA expression in malignant cells warrants further investigation.

It has been known for over twenty years that IFN can inhibit normal cell motility (Brouty-Boyé and Zetter, 1980), but the mechanism has not been identified. IFN has been used in the treatment of melanoma, renal cell carcinoma and other human neoplasms (Cascinelli et al., 2001; reviewed in Nanus, 2000; reviewed in Pastore et al., 2001). When expression of IFN-β was induced in PC-3M cells by transfection of an expression vector, these cells showed a reduced ability to metastasize and reduced tumorgenicity in nude mice (Dong et al., 1999). The authors demonstrated an anti-angiogenic effect of IFN on surrounding stroma. The data in the present report demonstrate that IFN directly inhibits PC-3M motility, indicating that IFN may affect both tumor and stroma. MxA is strongly induced by IFN, and MxA expression is a preferred marker for biologic evidence of IFN efficacy (Roers et al., 1994). Together, the data suggest that MxA may be a mediator of the effect of IFN on normal and tumor cell motility.

Motile cells are polarized, with a leading edge characterized by a ruffling lamellipodium and a trailing tail that retracts from substratum attachment sites. Actin polymerization is an essential force in cell propulsion, and actin-regulatory small G proteins regulate lamellipodia function. Our data demonstrate that MxA interacts with the microtubule cytoskeleton and that a point mutation of the MxA GTPase domain known to inactivate the GTPase (Ponten et al., 1997) also inactivates MxA control of motility and abolishes MxA association with microtubules. Studies of the cytoskeleton and motility have focused on actin and actin-regulatory small GTPases of the Rho family, rather than microtubules. Recent research, however, has demonstrated that in motile cells, microtubules regulate Rho protein activity and actin polymerization and, thus, microtubules are important regulators of directional movement (Waterman-Storer et al., 1999; Wittmann and Waterman-Storer, 2001). The data provided herein suggest that MxA may be a member of a new class of microtubule-associated proteins that regulate motility. Membrane ruffling, lamellipodium formation and mitosis appear uncompromised in time-lapse studies of MxA-expressing PC-3M cells. The pronounced decrease of vectorial movement despite unabated cell membrane activity suggests that MxA targets specific processes regulating motility, such as cell polarization and/or detachment from substratum adhesion sites (Ballestrem et al., 2000; Wittmann and Waterman-Storer, 2001).

A goal of the inventors was to identify a new pathway for the control of tumor cell motility and metastasis. This was achieved by the identification of MxA as a metastasis control gene. The level of MxA expression may be a predictor of metastatic potential. If this is verified, MxA could form a metastasis-specific component of the molecular phenotype and have an important impact on therapeutic decisions (Oh and Kantoff, 1999). Independent of its predictive value, the data presented here point to MxA as a new therapeutic target. Various expression strategies have been used to successfully identify tumor differentiation states, patient outcome and new therapeutic targets (Reiter et al., 1998; Yang et al., 1998; Amara et al., 2001; Argani et al., 2001; Saffran et al., 2001; Singh et al., 2002). The results of the present study, however, are particularly amenable to clinical translation, because the gene identified is highly inducible. Currently, only certain viruses or interferon have been shown to induce MxA expression. To develop an MxA-targeted small molecule, we are employing an MxA promoter-reporter system in a high throughput format to screen for inducers of MxA expression.

### Example 4

### Identification of Compounds that Induce MxA Expression

This Example provides an assay for quickly and easily screening for compounds and other agents that can increase MxA expression.

### Materials and Methods

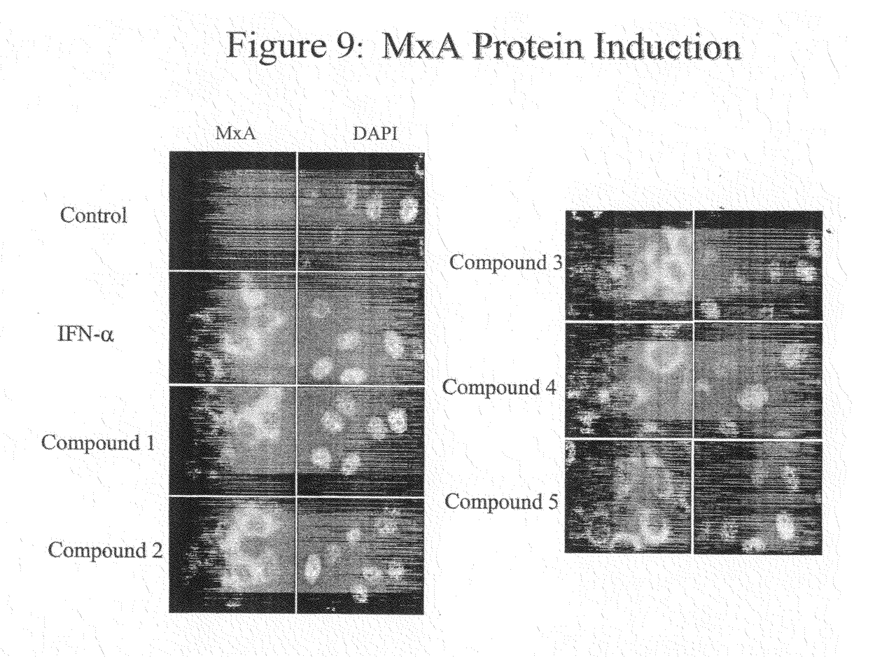

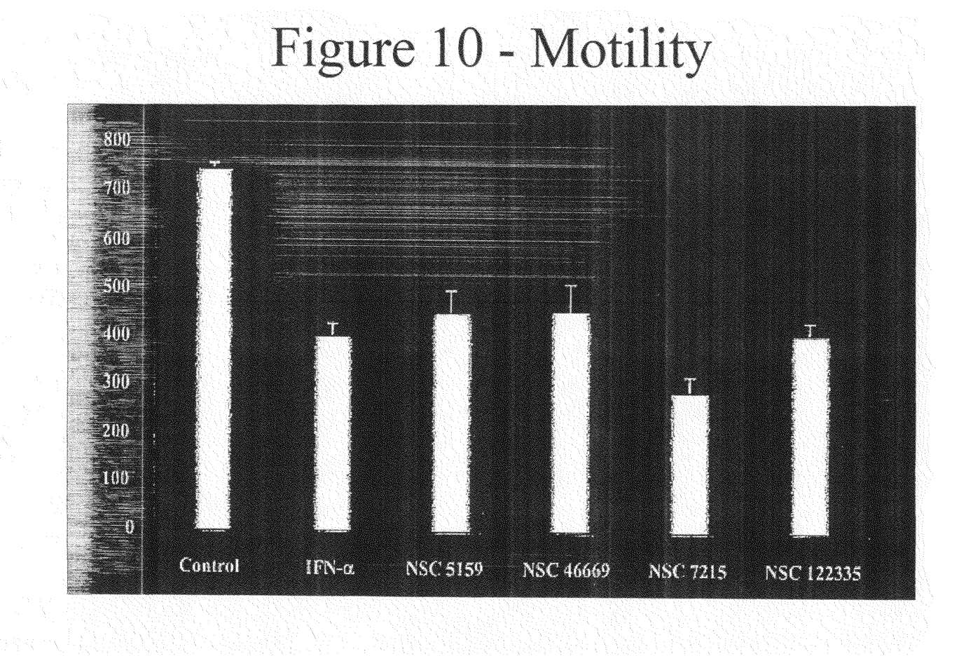

To develop a reporter system for drug screening, particularly for a high throughput assay procedure, the MxA promoter was cloned upstream of a nucleic acid segment that encoded luciferase (see FIG. 7). A nucleic acid encoding a eukaryotic selection marker (neomycin) was also inserted in the vector used for the reporter cassette. After transfecting this construct into human prostate carcinoma cells, a cell line that stably maintained the MxA reporter gene was cloned. Test agents, including a library of small molecules (the DTP 1900 chemotype library) were screened to ascertain whether any of the test agents increased luciferase expression. Of the 1900 compounds tested, 5 compounds activated the MxA promoter and increased luciferase expression. FIG. 8 illustrates that compounds NSC 34444, NSC 5159, NSC 46669, NSC 7215 and NSC 122335 induce expression of luciferase from the MxA promoter in PC-3M cells.

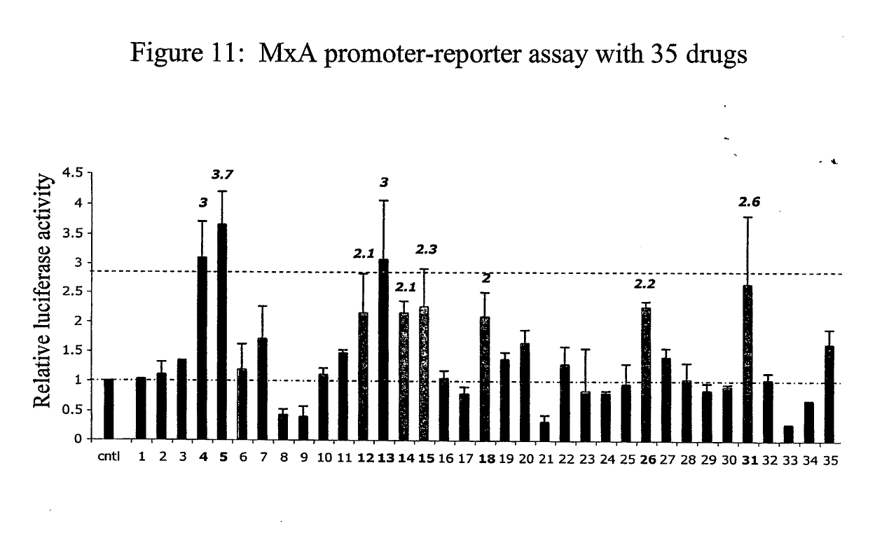

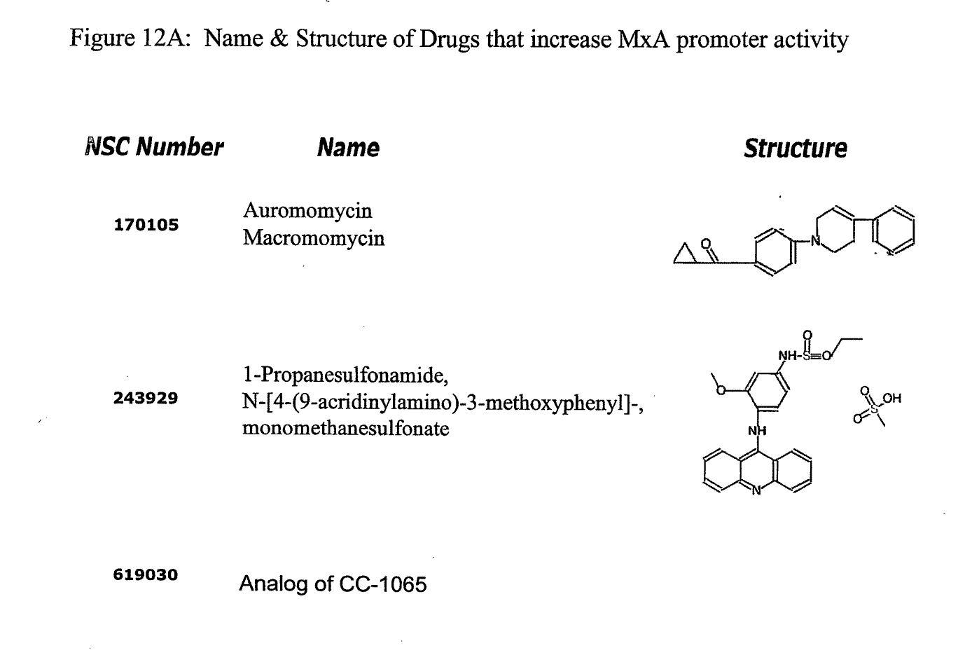

The five compounds that induced luciferase expression from the MxA were then tested to ascertain whether these compounds could also induce MxA protein expression and inhibit motility of PC-3-M prostate cancer cells. Each of the compounds could do so (FIGS. 9 and 10). FIG. 10 provides representative results illustrating that compounds NSC 5159, NSC 46669, NSC 7215 and NSC 122335 decrease the motility of PC-3M cells.

The five active compounds that can induce MxA expression and thereby reduce motility and metastasis of cancer cells have the following structures.

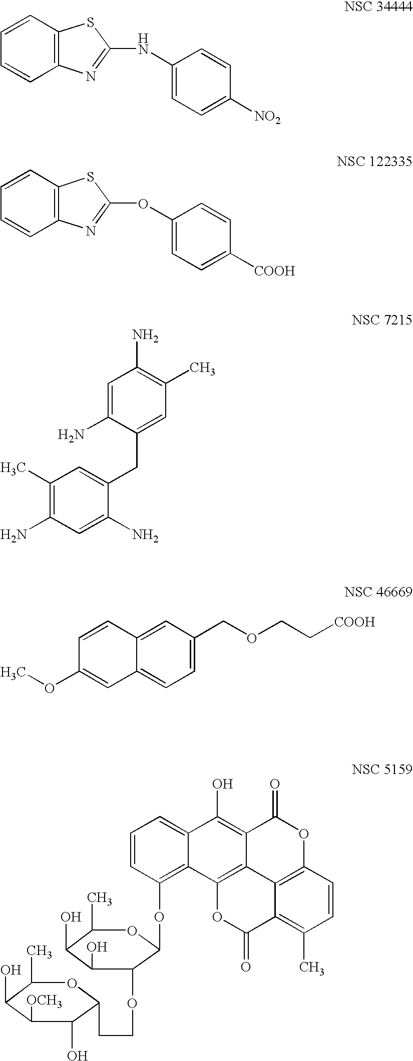

### Example 5

### Further Identification of Compounds that Induce MxA Expression

This Example provides additional screening results for identifying compounds and other agents that can increase MxA expression.

### Materials and Methods

Methods similar to those described in Example 4 were used to identify compounds that activate MxA expression.

### Results:

As shown in Table 1, of the 35 compounds tested at least nine activate the MxA promoter greater than 2-fold.

TABLE 1
Fold Increase in MxA Expression by Various Compounds

| Compound No. | NSC No. | Fold Increase |
|---|---|---|
| 1 | Control | 1.00 |
| 2 | 92498 | 1.03 |
| 3 | 140911 | 1.11 |
| 4 | 166381 | 1.32 |
| 5 | 170105 | 3.09 |
| 6 | 243929 | 3.65 |
| 7 | 270101 | 1.18 |
| 8 | 314622 | 1.71 |
| 9 | 325319 | 0.43 |
| 10 | 330500 | 0.40 |
| 11 | 338947 | 1.11 |
| 12 | 359079 | 1.46 |
| 13 | 376254 | 2.15 |
| 14 | 619030 | 3.07 |
| 15 | 635930 | 2.15 |
| 16 | 639831 | 2.25 |
| 17 | 641440 | 1.05 |
| 18 | 642035 | 0.79 |
| 19 | 645829 | 2.08 |
| 20 | 651649 | 1.37 |
| 21 | 651651 | 1.64 |
| 22 | 653629 | 0.32 |
| 23 | 665155 | 1.28 |
| 24 | 669705 | 0.84 |
| 25 | 678145 | 0.81 |
| 26 | 681284 | 0.95 |
| 27 | 687806 | 2.25 |
| 28 | 687810 | 1.40 |
| 29 | 690111 | 1.03 |
| 30 | 692406 | 0.85 |
| 31 | 692407 | 0.91 |
| 32 | 699152 | 2.64 |
| 33 | 699782 | 1.03 |
| 34 | 708444 | 0.27 |
| 35 | 715435 | 0.68 |
| 36 | 720444 | 1.62 |

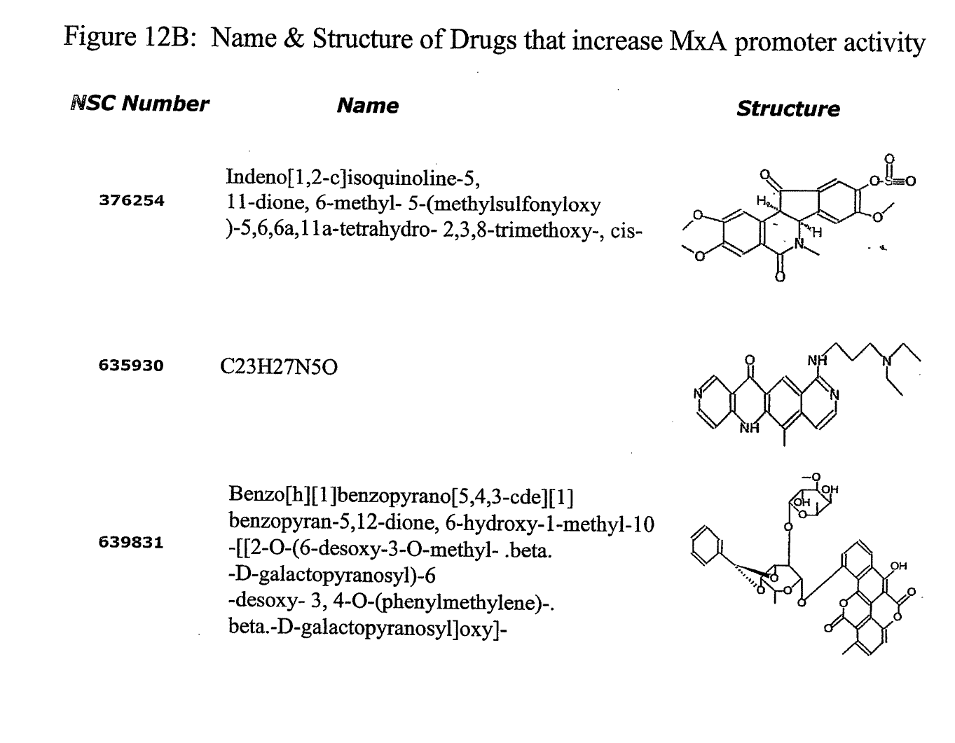

These results are graphically presented in FIG. 11, where the compound numbers given on the x-axis are the compound numbers provided in Table 1.

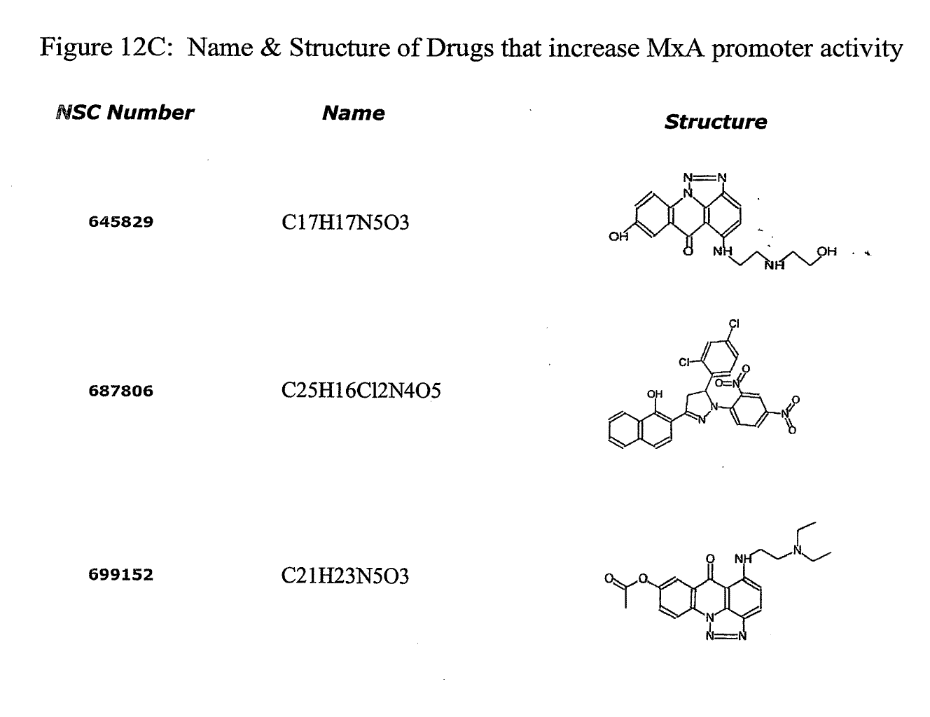

FIG. 12A-C provide structures for nine of the compounds listed in Table 1 that provide the greatest stimulation of MxA expression.

### Example 6

### Increased MxA Expression in Tumor Cells Increases Survival of Mice

This Example shows that mice injected with tumor cells live longer if those tumor cells express increased levels of MxA.

### Materials and Methods

The inventors have developed PC-3-M human prostate cancer cell lines that form tumors that metastasize in vivo. The PC-3-M prostate cancer cells also stably express luciferase at very high levels in vivo. Hence, PC-3-M cells are readily used for real-time in vivo analysis of tumor cell growth and metastasis.

In this Example, the effects of MxA expression in tumors formed by PC-3-M cells was examined to determine whether mice receiving PC-3-M cells that over-express MxA survive longer than mice who received PC-3-M cells that do not over-express MxA. Mice were injected intrasplenically with PC-3-M cells stably expressing neo-luciferase (PC-3-M-neo-luc), or with PC-3-M cells stably expressing MxA-luciferase (PC-3-M-MxA-luc). Morbidity-free survival was assessed non-invasively over a period of 40 days using Xenogen technology.

### Results

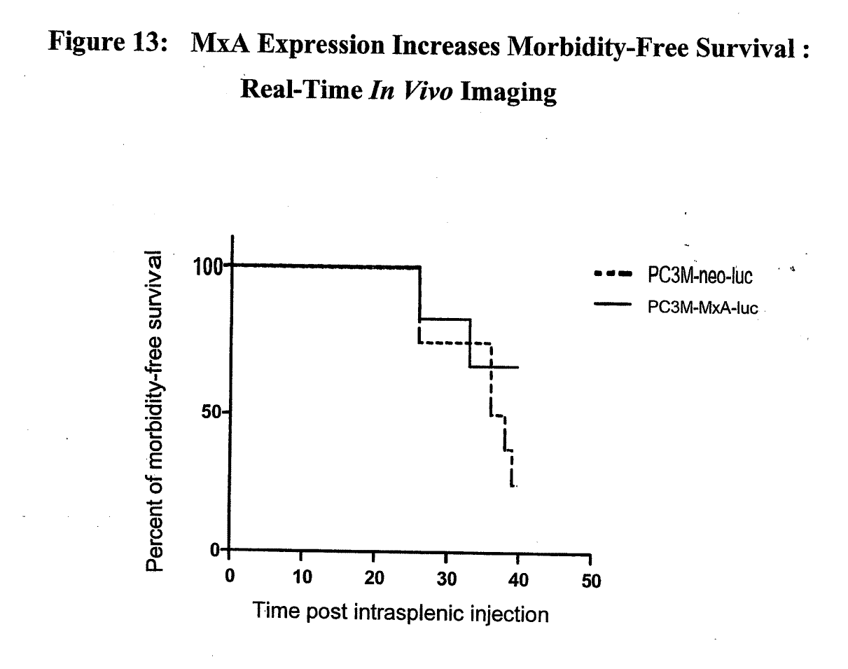

As shown in FIG. 13, mice who received PC-3-M tumors that over-express MxA survive longer than mice who received PC-3-M cells that do not over-express MxA.

### REFERENCES

al-Masri, A. N., Werfel, T., Jakschies, D. and von Wussow, P. (1997) Intracellular staining of Mx proteins in cells from peripheral blood, bone marrow and skin. Mol. Pathol. 50, 9-14.
Amara, N., Palapattu, G. S., Schrage, M., Gu, Z., Thomas, G. V., Dorey, F., Said, J., and Reiter, R. E. (2001) Prostate stem cell antigen is overexpressed in human transitional cell carcinoma. Cancer Res. 61, 4660-4665.
Argani, P., Rosty, C., Reiter, R. E., Wilentz, R. E., Murugesan, S. R., Leach, S. D., Ryu, B., Skinner, H. G., Goggins, M., Jaffee, E. M., Yeo, C. J., Cameron, J. L., Kern, S. E. and Hruban, R. H. (2001) Discovery of new markers of cancer through serial analysis of gene expression: prostate stem cell antigen is overexpressed in pancreatic adenocarcinoma. Cancer Res. 61, 4320-4324.
Ballestrem, C., Wehrle-Haller, B., Hinz, B., and Imhof, B. A. (2000) Actin-dependent lamellipodia formation and microtubule-dependent tail retraction control-directed cell migration. Mol. Biol. Cell 11, 2999-3912.
Bang, Y. J., Pirnia F, Fang, W. G., Kang, W. K., Sartor, O., Whitesell, L., Ha, M. J., Tsokos, M., Sheahan, M. D., Nguyen, M.-P., Niklinski, W. T., Myers, C. E., and Trepel, J. B. (1994) Terminal neuroendocrine differentiation of human prostate carcinoma cells in response to increased intracellular cyclic AMP. Proc. Natl. Acad. Sci. USA. 91, 5330-5334.
Brouty-Boyé, D. and Zetter, B. R. (1980) Inhibition of cell motility by interferon. Science 208, 516-518.
Cascinelli, N., Belli F., MacKie, R. M., Santinami, M., Bufalino, R., and Morabito, A. (2001) Effect of long-term adjuvant therapy with interferon alpha-2a in patients with regional node metastases from cutaneous melanoma: a randomized trial. Lancet 358, 866-869.
Choi, Y. H., Lee, S. J., Nguyen, M.-P., Jang, Y. S., Lee J., Wu, M. L., Takano, E., Maki, M., Henkart, P. A., and Trepel, J. B. (1997) Regulation of cyclin D1 by calpain protease. J. Biol. Chem. 272, 28479-28484.
Dong, A., Greene, G., Pettaway, C., Dinney, C. P. N., Eue, I., Lu, W., Bucana, C. D., Balbay, M. D., Bielenberg, D., and Fidler, I. J. (1999) Suppression of Angiogenesis, Tumorigenicity, and Metastasis by Human Prostate Cancer Cells Engineered to Produce Interferon-β. Cancer Res. 59, 872-879.
Fodstad, O., Aamdal, S., McMenamin, M., Neslad, J. M. and Pihl, A. (1988) A new experimental metastasis model in athymic nude mice, the human malignant melanoma LOX. Int. J. Cancer 41, 442-449.
Fort, P., Marty, L., Piechaczyk, M., Sabrouty, S., Dani, C., Jeanteur, P., and Blanchard, J. M. (1985) Various rat adult tissues express only one major mRNA species from the glyceraldehyde-3-phosphate-dehydrogenase multigene family. Nucleic Acids Res. 13, 1431-1442.
Goetschy, J. F., Zeller, H., Content, J., and Horisberger, M. A. (1989) Regulation of the interferon-inducible IFI-78K gene, the human equivalent of the murine Mx gene, by interferons, double-stranded RNA, certain cytokines, and viruses. J. Virol. 63, 2616-2622.
Hamdy, F. C. (2001) Prognostic and predictive factors in prostate cancer. Cancer Treat. Rev. 27, 143-151.
Hartwig, J. H. (1992) Mechanisms of actin rearrangements mediating platelet activation. J. Cell Biol. 118, 1421-1442.
Horisberger, M. A., and Hochkeppel, H. K. (1987) IFN-α induced human 78-kD protein: purification and homologies with the mouse Mx protein, production of monoclonal antibodies, and potentiation effect of IFN-α. J. Interferon Res. 7, 331-343.
Horisberger, M. A., McMaster, G. K., Zeller, H., Wathelet, M. G., Dellis, J., and Content, J. (1990) Cloning and sequence analyses of cDNAs for interferon- and virus-induced human Mx proteins reveal that they contain putative guanine nucleotide-binding sites: Functional study of the corresponding gene promoter. J. Virol. 64, 1171-1181.
Horisberger, M. A. (1992) Interferon-induced human protein MxA is a GTPase which binds transiently to cellular proteins. J. Virol. 66, 4705-4709.
Horisberger, M. A. (1995) Interferons, Mx genes, and resistance to influenza virus. Am. J. Respir. Crit. Care Med. 15Z S67-S71.
Khanna, C., Prehn, J., Yeung, C, Caylor, J, Tsokos, M., and Helman, L. (2000) An orthotopic model of murine osteosarcoma with clonally related variants differing in pulmonary metastatic potential. Clin. Exp-Metastasis 18, 261-271.
Kozlowski, J. M., Fidler, I. J., Campbell, D., Xu, Z., Kaighn, E., and Hart, I. R. (1984) Metastatic behavior of human tumor cell lines grown in the nude mouse. Cancer Res. 44, 3522-3529.
Lee, S. J., Ha, M. J., Lee, J., Nguyen, P., Choi, Y. H., Pimia, F., Kang, W. K., Wang, X. F., Kim, S. J., Trepel, J. B. (1998) Inhibition of the 3-hydroxy-3-methylglutaryl-coenzyme A reductase pathway induces p53-independent transcriptional regulation of p21(WAF1/CIP1) in human prostate carcinoma cells. J. Biol. Chem. 273, 10618-10623.
Liang, P., and Pardee, A. B. (1992) Differential display of eukaryotic mRNA by means of the polymerase chain reaction. Science 257, 867-971.
Nanus, D. M. (2000) New treatment approaches for metastatic renal cell carcinoma. Curr. Oncol. Rep. 2, 417-422.
Oh, W. K., and Kantoff, P. W. (1999) Treatment of locally advanced prostate cancer: is chemotherapy the next step? J. Clin. Oncol. 17, 3664-3675.
Pastore, R. D., Pfeffer, D. M., and Nanus, D. M. (2001) Renal cell carcinoma and interferon at the millenium. Cancer Invest. 19, 281-291.
Ponten, A., Sick, C., Weber, M., Haller, O., and Kochs, G. (1997) Dominant-negative mutants of human MxA protein: domains in the carboxy-terminal moiety are important for oligomerization and antiviral activity. J. Virol. 71, 2591-2599.
Reiter, R. E., Gu, Z., Watabe, T., Thomas, G., Szigeti, K., Davis, E., Wahl, M., Nisitani, S., Yamashiro, J., Le Beau, M. M., Loda, M., and Witte, O. N. (1998) Prostate stem cell antigen: a cell surface marker overexpressed in prostate cancer. Proc. Natl. Acad. Sci. U.S.A. 95, 1735-1740.
Roers, A., Hochkeppel, H. K., Horisberger, M. A., Hovanessian, A., and Haller, O. (1994) MxA gene expression after live virus vaccination: a sensitive marker for endogenous type I interferon. J. Infect. Dis. 169, 807-813.
Ronni, T., Matikainen, S., Lehtonen, A., Palvimo, J., Dellis, J., Van Eylen, F., Goetschy, J. F., Horisberger, M., Content, J., and Julkunen, I. (1998) The proximal interferon-stimulated response elements are essential for interferon responsiveness: a promoter analysis of the antiviral MxA gene. J. Interferon Cytokine Res. 18, 773-781.
Saffran, D. C., Raitano, A. B., Hubert, R. S., Witte, O. N., Reiter, R. E., and Jakobovits, A. (2001) Anti-PSCA mAbs inhibit tumor growth and metastasis formation and prolong the survival of mice bearing human prostate cancer xenografts. Proc. Natl. Acad. Sci. U.S.A. 98, 2658-2663.
Sambrook, J., Fritsch, E. F., and Maniatis, T. (1989) Molecular cloning. A laboratory manual, 2^{nd} Ed. Cold Spring Harbor, N.Y.
Scherf, U., Ross, D. T., Waltham, M., Smith, L. H., Lee, J. K., Tanabe, L., Kohn, K. W., Reinhold, W. C., Myers, T. G., Andrews, D. T., Scudiero, D. A., Eisen, M. B., Sausville, E. A., Pommier, Y., Botstein, D., Brown, P. O., and Weinstein, J. N. (2000) A gene expression database for the molecular pharmacology of cancer. Nat. Genet. 24, 236-244.
Singh, D., Febbo, P. G., Ross, K., Jackson, D. G., Manola, J., Ladd, C., Tamayo, P., Renshaw, A. A., D'Amico, A. V., Richie, J. P., Lander, E. S., Loda, M., Kantoff, P. W., Golub, T. R., and Sellers, W. R. (2002) Gene expression correlates of clinical prostate cancer behavior. Cancer Cell 1, 203-209.
Tazi-Ahnini, R., di Glovine, F. S., McDonagh, A. J. G., Messenger, A. G., Amadou, C., Cox, A., Duff, G. W., and Cork, M. J. (2000) Structure and polymorphism of the human gene for the interferon-induced p78 protein (MY1): evidence of association with alopecia greata in the Down syndrome region. Hum. Genet. 106, 639-645.
Van der Bliek, A. (1999) A functional diversity in the dynamin family. Trends Cell Biol. 9, 96-102.
Waterman-Storer, C. M., Worthylake, R. A., Liu, B. P., Burridge, K., and Salmon, E. D. (1999) Microtubule growth activates RacI to promote lamellipodial protrusion in fibroblasts. Nat. Cell Biol. 1, 45-50.
Wittmann, T. and Waterman-Storer, C. M. (2001) Cell motility: can Rho GTPases and microtubules point the way? J. Cell Sci. 114, 3795-3803.
Yamada, T., Horisberger, M. A., Kawaguchi, N., Moroo, I., and Toyoda, T. (1994) Immunohistochemistry using antibodies to alpha-interferon and its induced protein, MxA, in Alzheimer's and Parkinson's disease brain tissues. Neurosci. Lett. 181, 61-64.
Yang, R. M., Naitoh, J., Murphy, M., Wang, H. J., Phillipson, J., deKernion, J. B., Loda, M., and Reiter, R. E. (1998) Low p27 expression predicts poor disease-free survival in patients with prostate cancer. J. Urol. 159, 941-945.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of treating or preventing cellular migration in a mammal comprising administering to the mammal an effective amount of an agent that increases the expression or activity of MxA in the mammal.

2. The method of claim 1, wherein the agent is a benzopyrene saccharide or a compound of formula I, or a pharmaceutically acceptable salt thereof.
R₁—X(R₃)—R₂ I
wherein:
X is methylene (CH₂), nitrogen or oxygen;
R₁ and R₂ are cycloalkyl, aryl, arylalkylene, heteroaryl, heterocyclyl, or alkyl, any of which may be substituted with oxygen (O), hydroxyl (OH), sulfite (SO₃), sulfate (SO₄), sulfonamide (NH—SO₂ or NH—SO₃), halogen (F, Cl, Br, or I), carboxylate (CO₂), nitro (NO₂), amino (NH₂), secondary or tertiary alkylamino, alkylsulfonamide, lower alkyl, cycloalkyl, alkylenehydroxy, alkoxy, alkoxycarbonyl, alkoxyalkylenecarboxylic acid, alkylenecarboxylic acid, alkyleneaminoalkylene, alkyleneaminoalkylenehydroxy, alkanoyloxy, aminoaryl or aryl; and
R₃ is nothing, hydrogen or, together with an X nitrogen to which it is attached, forms a heterocyclic ring with 0-2 double bonds between the carbon atoms of the heterocyclic ring or 0-1 additional nitrogen atoms.

3. The method of claim 2, wherein the compound of formula I is any one of the following compounds, or a combination thereof:

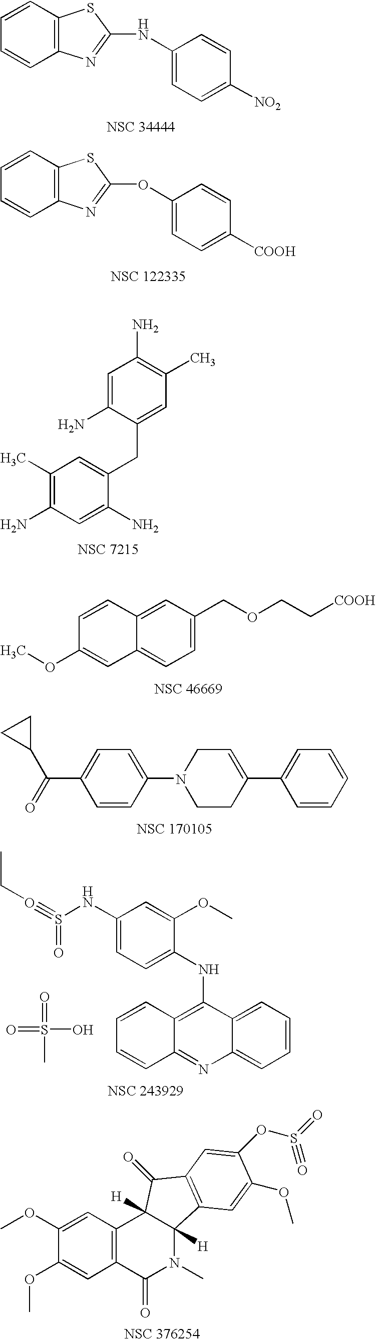

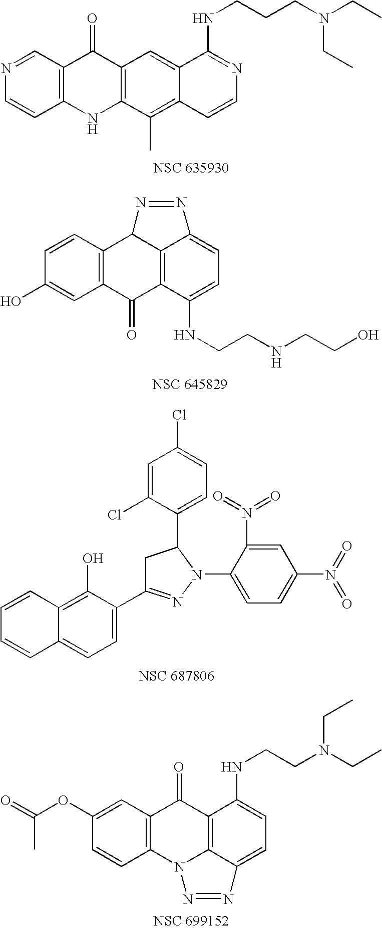

4. The method of claim 2, wherein the benzopyrene saccharide is one of the following compounds, or a combination thereof:

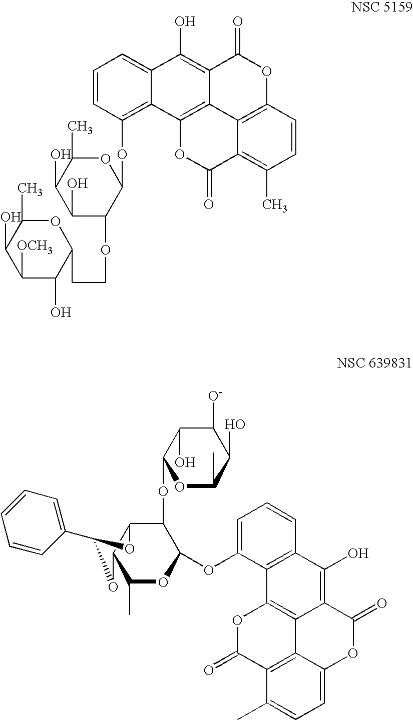

5. The method of claim 1, wherein the agent is a compound selected from NSC 34444, NSC 122335, NSC 46669, NSC 7215, or NSC 5159.

6. The method of claim 1, wherein the agent is one of the following compounds, or a combination thereof:

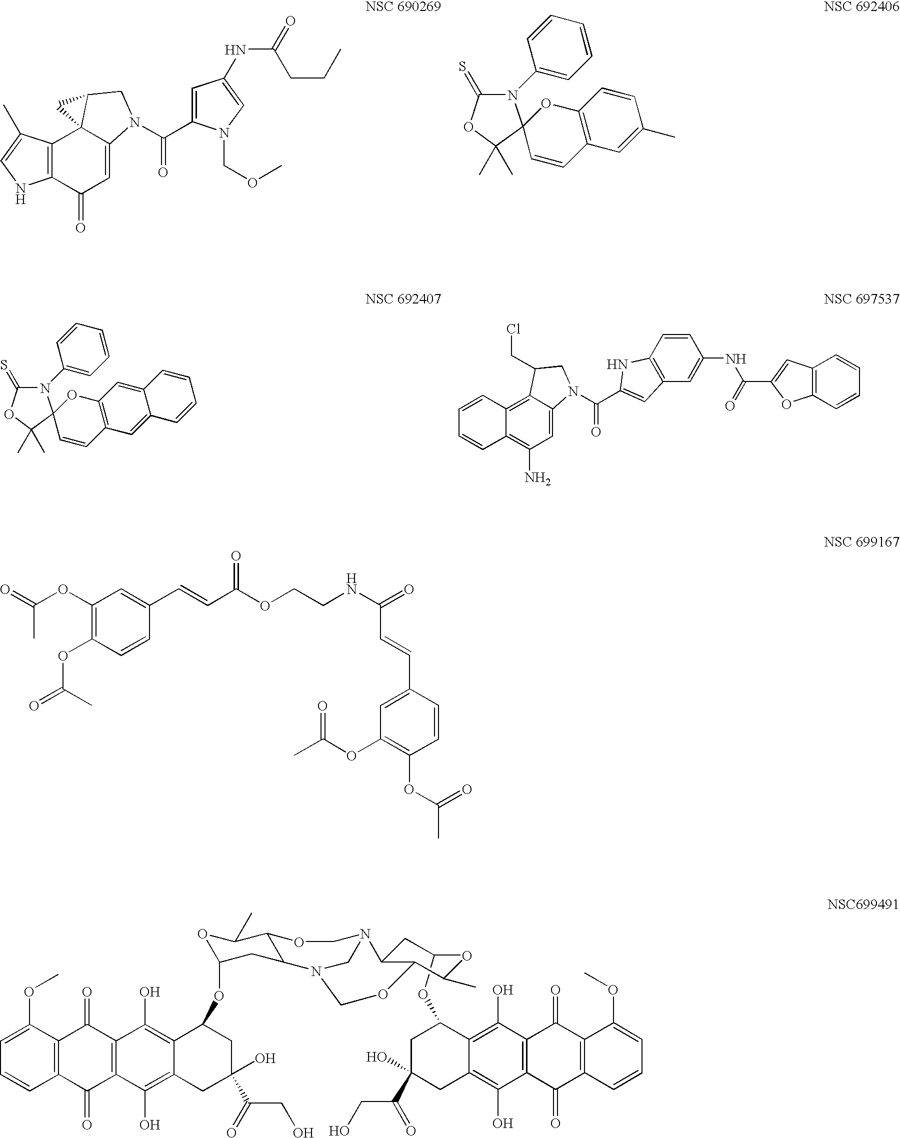

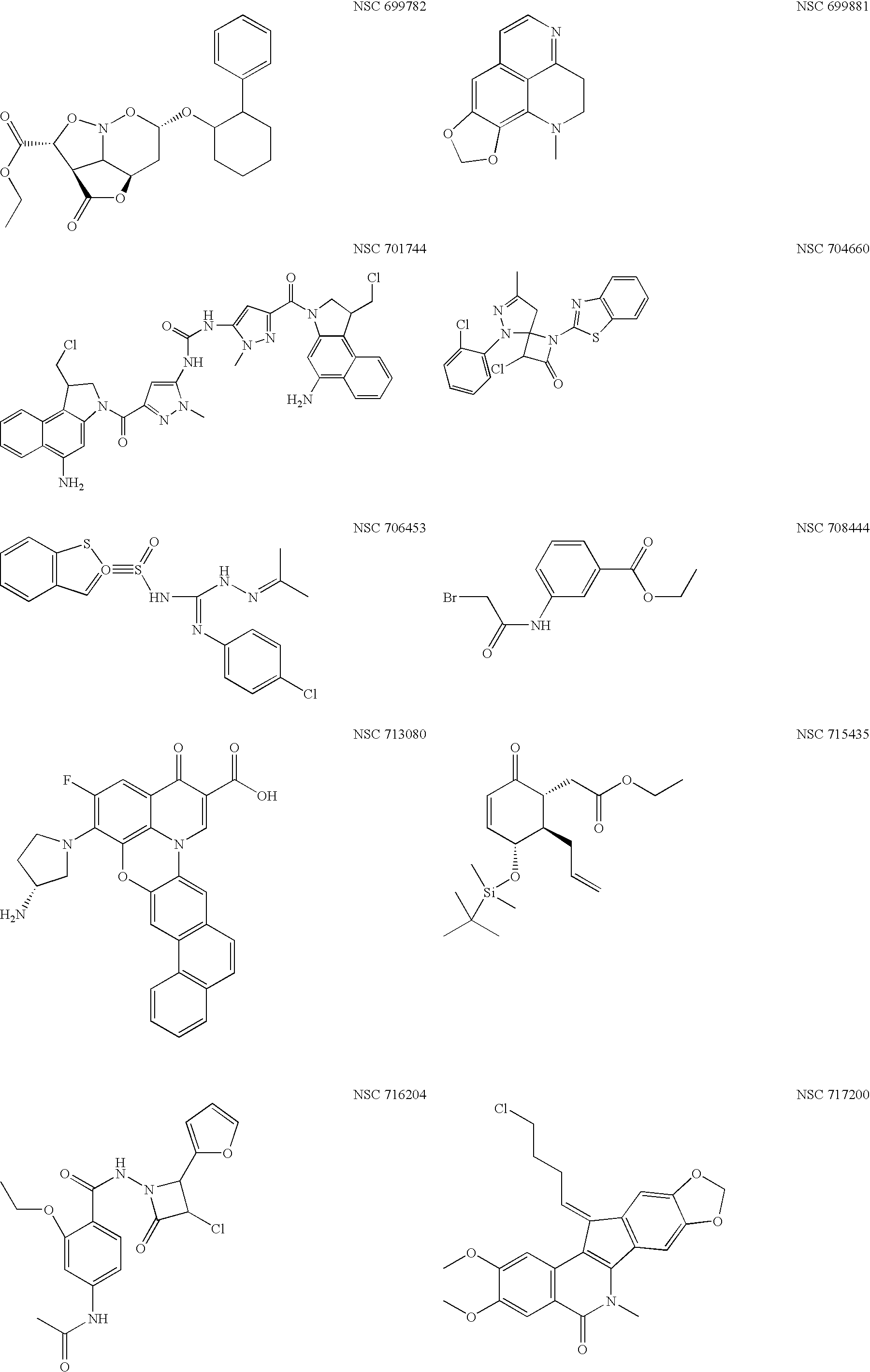

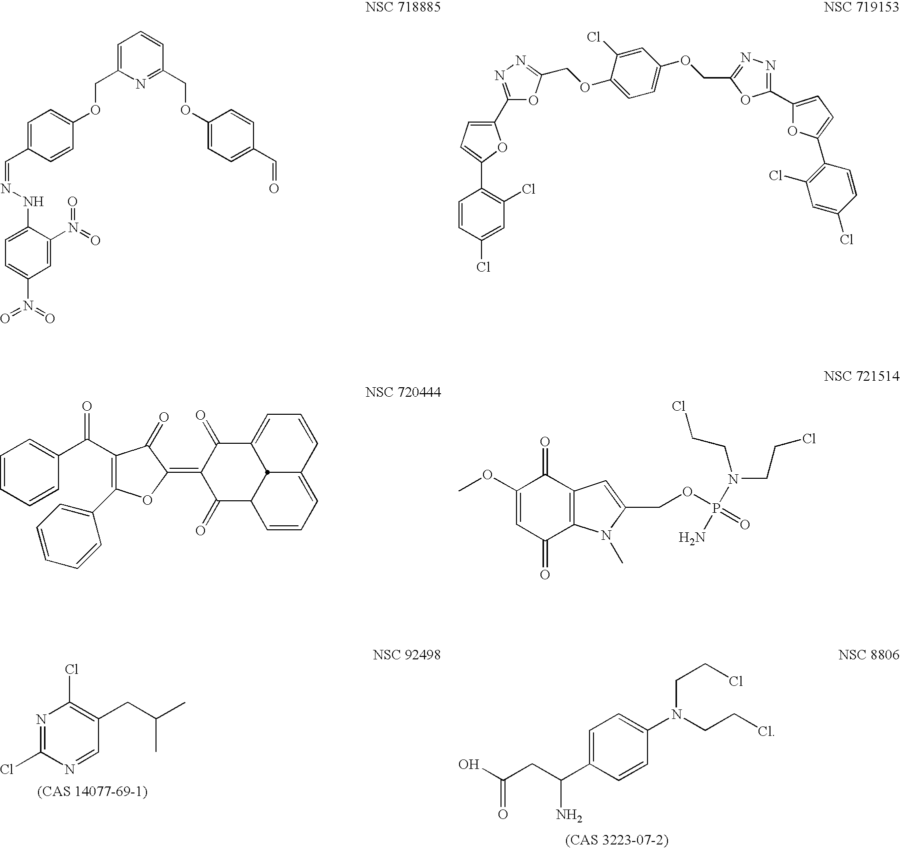

7. The method of claim 1, wherein the mammal is a human.

8. The method of claim 1, wherein the cellular migration is cancer cell metastasis.

9. The method of claim 8, wherein the cancer is prostate cancer.

10. The method of claim 8, wherein the cancer is a carcinoma.

11. The method of claim 8, wherein the cancer is an adenocarcinoma.

12. The method of claim 8, wherein the cancer is cancer of a breast, bladder, colon, kidney, liver, lung, esophagus, gall-bladder, ovary, pancreas, stomach, cervix, thyroid, prostate, skin, central nervous system or peripheral nervous system tissue.

13. The method of claim 1, wherein the agent comprises a MxA polypeptide.

14. The method of claim 13, wherein the MxA polypeptide comprises SEQ ID NO:1.

15. The method of claim 13, wherein the MxA polypeptide further comprises a protein transduction domain selected from the group consisting of SEQ ID NO:4, SEQ ID NO:5, SEQ ID NO:6 and SEQ ID NO:7.

16. The method of claim 1, wherein the agent is a MxA nucleic acid encoding a MxA polypeptide, wherein the MxA nucleic acid is operably linked to a promoter that can effect expression of the MxA polypeptide.

17. The method of claim 16, wherein the MxA nucleic acid comprises SEQ ID NO:2.

18. The method of claim 1, wherein the effective amount is a therapeutically effective amount.

19. The method of claim 1, wherein the agent is administered locally to a tumor.

20. A method of identifying an agent that inhibits metastatic cancer comprising contacting a cancer cell with a test agent, observing whether expression is increased from a MxA promoter within the cancer cell, and thereby identifying a test agent that inhibits metastatic cancer.

21. The method of claim 20, wherein the MxA promoter is linked to a nucleic acid encoding a reporter molecule.

22. The method of claim 20, wherein the reporter molecule is luciferase.

23. A method of identifying an agent that inhibits metastatic cancer in a mammal comprising:
(a) injecting the mammal with a tumor cell that comprises a first nucleic acid encoding a MxA promoter operably linked to a second nucleic segment encoding a reporter molecule;
(b) administering a test agent to the mammal; and
(c) observing whether tumor cells can be detected in the mammal at sites distance from the primary site of tumor cell injection;
wherein the tumor cell can form a metastiatic tumor in the mammal.

24. The method of claim 23, which further comprises quantifying expression of the reporter molecule in tumor cells at the primary site of tumor cell injection or in tumor cells at sites distance from the primary site of tumor cell injection.

25. The method of claim 23, wherein the reporter molecule is luciferase.

26. A method of treating viral infection in a mammal comprising administering to the mammal an effective amount of an agent that increases the expression or activity of MxA in the mammal.

27. The method of claim 26, wherein the agent is a benzopyrene saccharide or a compound of formula I, or a pharmaceutically acceptable salt thereof.
R₁—X(R₃)—R₂ I
wherein:
X is methylene (CH₂), nitrogen or oxygen;
R₁ and R₂ are cycloalkyl, aryl, arylalkylene, heteroaryl, heterocyclyl, or alkyl, any of which may be substituted with oxygen (O), hydroxyl (OH), sulfite (SO₃), sulfate (SO₄), sulfonamide (NH—SO₂ or NH—SO₃), halogen (F, Cl, Br, or I), carboxylate (CO₂), nitro (NO₂), amino (NH₂), secondary or tertiary alkylamino, alkylsulfonamide, lower alkyl, cycloalkyl, alkylenehydroxy, alkoxy, alkoxycarbonyl, alkoxyalkylenecarboxylic acid, alkylenecarboxylic acid, alkyleneaminoalkylene, alkyleneaminoalkylenehydroxy, alkanoyloxy, aminoaryl or aryl; and
R₃ is nothing, hydrogen or, together with an X nitrogen to which it is attached, forms a heterocyclic ring with 0-2 double bonds between the carbon atoms of the heterocyclic ring or 0-1 additional nitrogen atoms.

28. The method of claim 27, wherein the compound of formula I is any one of the following compounds, or a combination thereof:

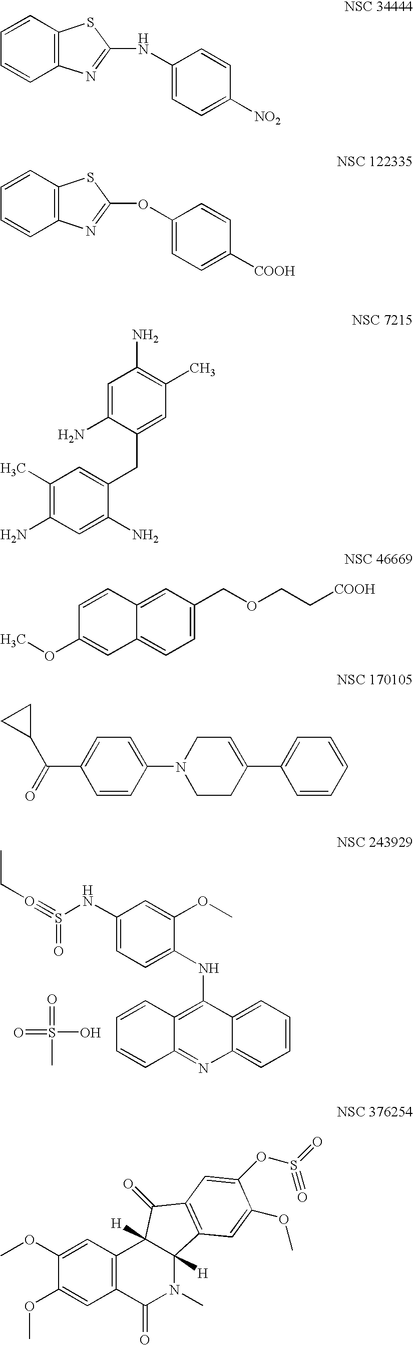

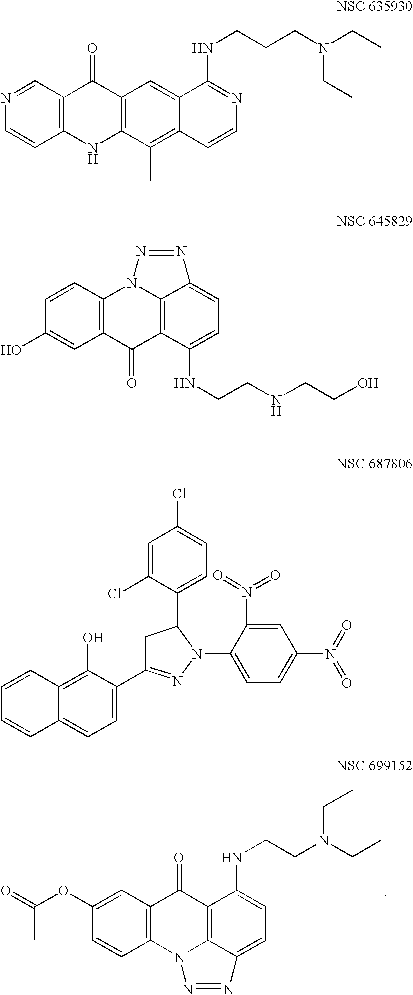

29. The method of claim 27, wherein the benzopyrene saccharide is one of the following compounds, or a combination thereof:

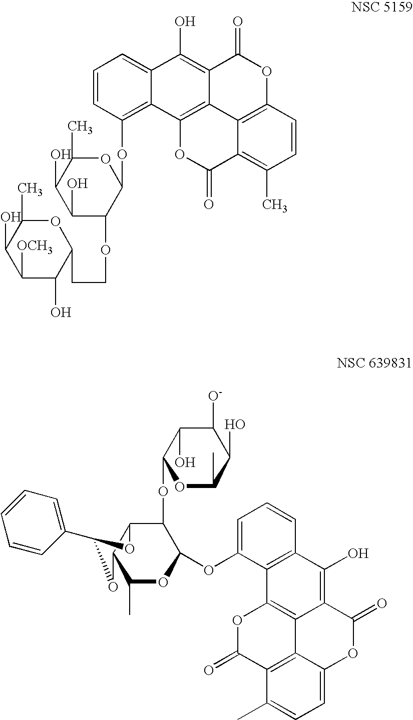

30. The method of claim 26, wherein the viral infection is an infection caused by an enveloped or non-enveloped virus.

31. The method of claim 26, wherein the viral infection is an infection caused by influenza virus type A and B, avian influenza (bird flu), avian influenza A (H5N1), hepatitis A virus, hepatitis B virus, hepatitis C virus, human immunodeficiency virus (HIV), poxvirus, herpes virus, adenovirus, papovavirus, parvovirus, reovirus, orbivirus, picornavirus, rotavirus, alphavirus, rubivirus, flavivirus, coronavirus, paramyxovirus, morbillivirus, pneumovirus, rhabdovirus, lyssavirus, orthmyxovirus, bunyavirus, phlebovirus, nairovirus, hepadnavirus, arenavirus, retrovirus, enterovirus, rhinovirus or filovirus.

32. The method of claim 26, wherein the viral infection is an infection caused by hemorrhagic fever virus, Chikungunya virus, Japanese encephalitis virus, Monkey pox virus, variola virus, Congo-Crimean haemorrhagic fever virus, Junin virus, Omsk haemorrhagic fever virus, Venezuelan equine encephalitis virus, Dengue fever virus, Lassa fever virus, Rift valley fever virus, SARS coronavirus, Western equine encephalitis virus, Eastern equine encephalitis virus, Lymphocytic choriomeningitis virus, Russian Spring-Summer encephalitis virus, White pox, Ebola virus, Machupo virus, Smallpox virus, Yellow fever virus, Hantaan virus, Marburg virus, or Tick-borne encephalitis virus.

33. A composition for treating or preventing cellular motility of a cell in a mammal comprising a therapeutically effective amount of an agent that increases the expression or activity of MxA in the mammal.

34. The composition of claim 33, wherein the agent is a benzopyrene saccharide or a compound of formula I, or a pharmaceutically acceptable salt thereof.
R₁—X(R₃)R₂ I
wherein:
X is methylene (CH₂), nitrogen or oxygen;
R₁ and R₂ are cycloalkyl, aryl, arylalkylene, heteroaryl, heterocyclyl, or alkyl, any of which may be substituted with oxygen (O), hydroxyl (OH), sulfite (SO₃), sulfate (SO₄), sulfonamide (NH—SO₂ or NH—SO₃), halogen (F, Cl, Br, or I), carboxylate (CO₂), nitro (NO₂), amino (NH₂), secondary or tertiary alkylamino, alkylsulfonamide, lower alkyl, cycloalkyl, alkylenehydroxy, alkoxy, alkoxycarbonyl, alkoxyalkylenecarboxylic acid, alkylenecarboxylic acid, alkyleneaminoalkylene, alkyleneaminoalkylenehydroxy, alkanoyloxy, aminoaryl or aryl; and
R₃ is nothing, hydrogen or, together with an X nitrogen to which it is attached, forms a heterocyclic ring with 0-2 double bonds between the carbon atoms of the heterocyclic ring or 0-1 additional nitrogen atoms.

35. The composition of claim 34, wherein the compound of formula I is any one of the following compounds, or a combination thereof:

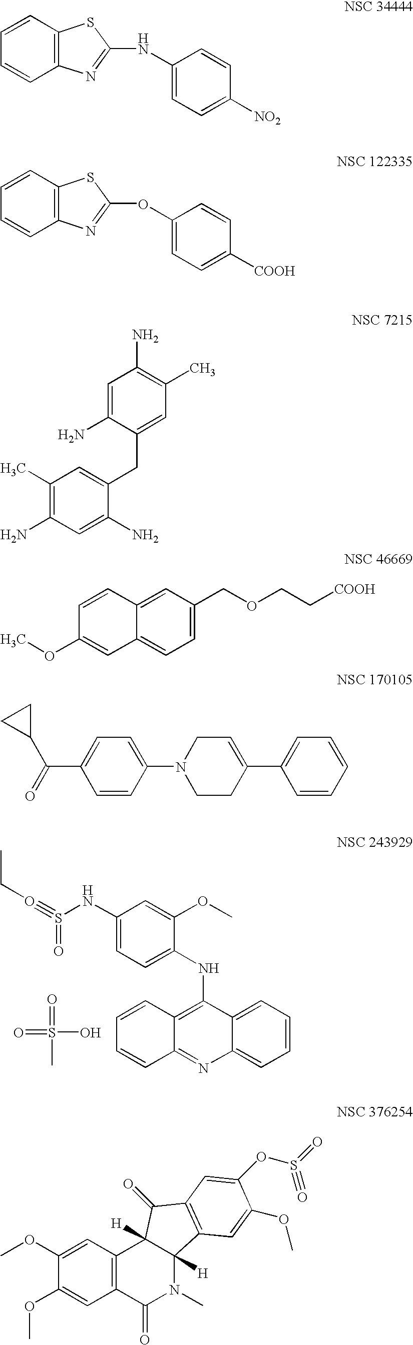

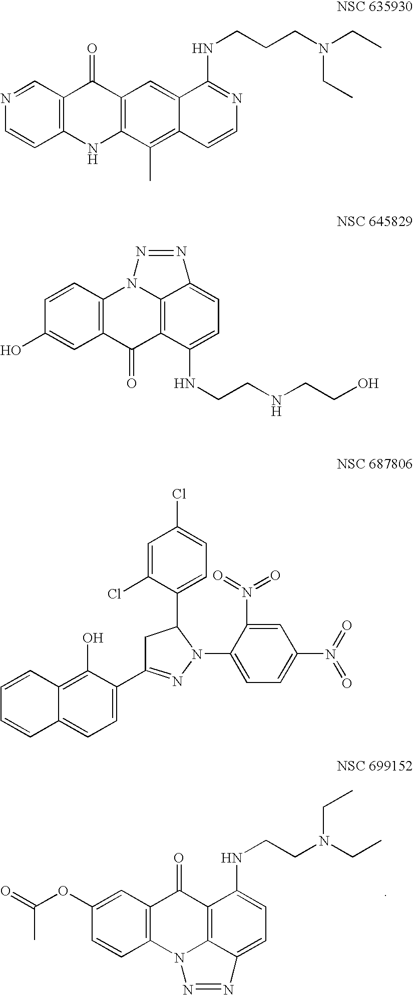

36. The composition of claim 34, wherein the benzopyrene saccharide is one of the following compounds, or a combination thereof:

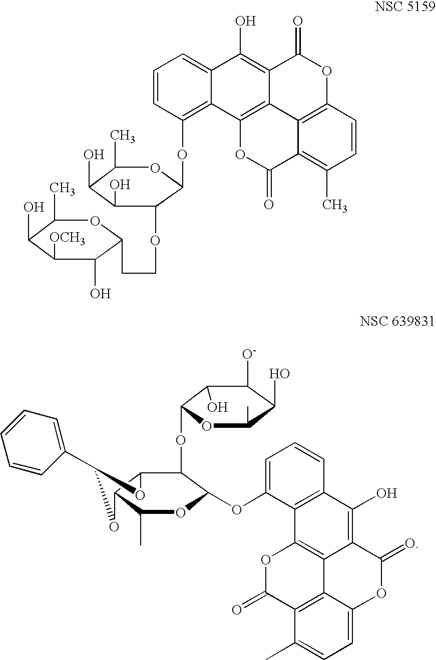

37. The composition of claim 33, wherein the agent is fusion protein comprising a MxA polypeptide and a protein transduction domain consisting of any one of SEQ ID NO:4-7.

38. The composition of claim 33, wherein the composition is formulated for local delivery to a tumor.

39. A fusion protein comprising a MxA polypeptide and a protein transduction domain consisting of any one of SEQ ID NO:4-7.

40. (canceled)


SEQUENCE LISTING (7 sequences)

SEQ ID NO: 1 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKINAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 2 - DNA, 2753 bp, Homo sapiens
ccacgcgtccgcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacattgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaaattaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccaatccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacaaaaaaaaaaaaaaaaaa

SEQ ID NO: 3 - DNA, 1899 bp, Homo sapiens
aagctttattattactattttatttattttattttattttccttccacacacccgtttccaccctggagaggccagatgagccagactccagggaggcctagaagtgggcaaggggaaacgggaaaggaggaagatggtatgggtgtgcctggttaggggtgggagtgctggacggagttcgggacaagaggggctctgcagcattgcacacaatgcctgggagtcctgctggtgctgggatcatcccagtgagccctgggagggaactgaagacccccaattaccaatgcatctgttttcaaaaccgacggggggaaggacatgcctaggttcaaggatacgtgcaggcttggatgactccgggccattagggagcctccggagcaccttgatcctcagactggcctgatgaaacgagcatctgattcagcaggcctgggttcgggcccgagaacctgcgtctcccgcgagttcccgcgaggcaagtgctgcaggtgcggggccaggagctaggtttcgtttctgcgcccggagccgccctcagcacagggtctgtgagtttcatttcttcgccggcgcgggcggggctgggcgcggggtgaaagaggcgaacgagagcggaggccgcactccagcactgcgcagggaccggtgagtgtcgcttctgggggcagcgcagtaaccgcgctaggagcgcgagaagggcattgggagagcggcgttcgtgcgagactagcgctccggagcacgggcacgacgggggcaccttctcggctgctagtaactaacaataataataatcataatcatagcaagggcgctgatgggcgggctcggagcacgcctgattctggttcccaccaggctgcccaggctcctgatgacgcatcagaaacatccccctaacccgcggccttcctgcaggagaggttgggaaggggtgggggacggggctcgggggaggtctccgagggactctagtaagcggggaagggcgccgggaaagtttcagatccacggtgcgcgggccacgagccacccgaacgccgaccactgctttccgtcgacttctatttcctgggaacgcgcgaaacaagacccaagtcagactgcggaggtcgctggggagggaaggttcaaggagttctcgccgatcctgctgaataaagggggttccgagctgggccgagatggggcatgcgcgggaagacccctgcccgctgttcccccccaccgccccagtggatgccatgcctggggctccccggcgcgtggggctgacgcaccctcgggtccatcgtagttggccggatcgtggagtgggtgcggtggacgaagggaggcaggacagtcccgggggtggcagaaggagcccgggcacagctgagacctgcgctcccatcccaccaacactcacagcaggtgctgccgagctgggcaattgggatggcccaagttatttggttaaattttaaatcacgtttgttactgggaagtagagtccagtgatgctaaccgcgcctctacctcaccaccggtgtcagtccaaagggctcctaaaatggctgtgtcatctttcagccttggaccgcagttgccggccaggaatcccagtgtcacggtggacacgcctccctcgcgcccttgccgcccacctgctcacccagctcaggggctttggtaggtagcagtgcatttggtctaaagggcaagatgttctctcttttattcataacaaatttaaataccagcagggtttggggggaaaaacgctttcagaagaaaaggtgaatgtcagtcctgcaagagttagttttaaaactagactgaattggcacatgtatacctatgtaacaaacctgcacgttctgcacatgtaccccagaacttaaaagctt

SEQ ID NO: 4 - protein, 18 aa, Human immunodeficiency virus-1
LGISYGRKKRRQRRRPPQ

SEQ ID NO: 5 - protein, 13 aa, Human immunodeficiency virus-1
GRKKRRQRRRPPQ

SEQ ID NO: 6 - protein, 11 aa, Human immunodeficiency virus-1
YGRKKRRQRRR

SEQ ID NO: 7 - protein, 16 aa, Drosophila
RQIKIWFQNRRMKWKK